SEQUENCE LISTING (13 sequences)

SEQ ID NO: 1 - protein, 892 aa, Lactococcus lactis
LFALDWKRIFKNPVATFLIVALMIIPSLYAWFNIKALWDPYANTSQLPIAVYSADKPASFQDKTINIGDEVLKNLKKNHQLGWKFVDSKAELDKGVKSGKYYAGIYLPKDFSKDLLSFTTGDIKKPQIVYSINEKINAIAPKITSKGASSLQSQISEEFINTASSTLLKTFNTIGYDIDKNMVSIQKVKSAILSTNDNLGTIDKYTQQVVDLHGKMPEIKEKLAKANEFITYLPEVDSLGQKVISLNDKMPEIDKSLSLVLTLQEKIPEIQNAGKQISMIDEDFASVESTMTQGIQEAKDGLQIINQVQKSMPDIQKLGQDADSLGTVTLDAAKKMQSALPSITNSVQITLQSIQEFSKNTSSVLAVIDQAIADNQLTDEEKEQINSLITNFINNIARQREAIQNIVDYMKQVQESNGSHDLDSTIEQLSNLDSLLSGLSTRMNHLNELVQNGDVSEIRAYLSQINDVVTKISNLVDNIDVSGISKTIDTVLTSLTKTIADAKGLLNQAQQIDFEALLNSTSQTVSNAVTILEKYQGEMPAIKQEIHDANLMLNGNMTTIVNAINEGASLYKNEFPILKTKLGTASDFFKNDYAGVRKDLTDTLTMANEKMPDVESALNQANSLIQNDWPEIKSGIQKAATAIQKGEKEVDLGEIVKLLKLDATKESDFWTQPVEVQENQVYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIIFVAIGAILFPHFEDKTKKISEIGSKSHFFH

SEQ ID NO: 2 - DNA, 2679 bp, Lactococcus lactis
ttgtttgccctagactggaaacgaatttttaaaaatcctgtggcgacctttttaatcgttgccttaatgataattccctctttatatgcttggtttaacattaaagccttgtgggacccttatgccaatacctcgcaattgccaattgcagtctacagtgcagataaacctgctagttttcaagataaaaccataaacattggtgatgaagttctgaagaatttaaagaaaaatcatcaattaggttggaaatttgtagattctaaagcagaattagataaaggagttaaatccggaaaatactatgctggaatttacttaccaaaggatttttcaaaagaccttttaagttttacaaccggtgatattaaaaagccacaaatagtttattctattaatgaaaagataaatgctattgctcccaaaataacctctaagggtgcttcatctcttcaatcacagatttctgaagagtttattaatacggcaagtagtacactcttaaaaacctttaatacgattggttacgatattgataaaaatatggtcagcattcaaaaggttaagtcagcaattttatcaacaaacgataatctaggaaccattgacaaatacacccaacaagtggttgaccttcatggtaaaatgccagaaatcaaagaaaaactggctaaggctaacgaatttattacttatttacccgaagttgattctttaggtcaaaaagtaattagtttgaatgataaaatgcctgaaattgataaaagtttgtcacttgttttgactcttcaagaaaaaattcctgaaattcaaaacgctggtaaacaaatttcgatgattgatgaagactttgcgtcagttgaaagtactatgacccaaggaattcaagaagcaaaagacggcttgcaaatcatcaatcaagtccaaaaatcaatgccagacattcaaaaattaggacaagatgcagatagtttagggactgtgacattggatgcagcgaaaaaaatgcagagcgcattgcctagtatcacgaatagcgttcaaatcactctacagtctattcaagaattttcaaaaaatacttcctcagttctcgctgttattgaccaagcaatagctgataatcagctcacagatgaagaaaaagaacaaataaatagtttaattaccaactttattaacaatatcgcccgacaaagagaagcaattcaaaatattgttgattacatgaaacaagtccaagagagcaatggaagtcatgatttagactcaacaattgagcaactcagtaatcttgattctttactgtcaggacttagtactagaatgaatcatctgaatgagttagtacaaaatggtgatgtatctgaaattcgtgcttacttatcccaaattaatgatgtggttactaaaatctctaatttagttgataatattgatgtgagtggaattagtaaaacaattgatacagttttaacaagcttgactaaaacaatcgctgatgccaaaggtttattaaatcaagctcaacaaattgactttgaagctttactaaattcaacaagtcaaacagtttctaatgctgttaccattttagaaaaatatcaaggtgaaatgccagccatcaaacaagaaattcatgatgcaaacttgatgttaaatggaaatatgacgacaattgtcaatgcaattaacgaaggggctagtctttataaaaatgagttccctatccttaaaactaagttgggcacagcttctgatttctttaaaaatgattatgctggtgttcgtaaagatttaacagataccttaacaatggcaaatgagaaaatgcctgacgtcgagtctgctttgaatcaagccaatagtttaatccaaaatgactggcctgaaatcaaatcgggcattcaaaaagcagcaacagccattcaaaaaggtgaaaaagaagttgatttaggagagattgttaaacttctaaaacttgatgctactaaagagagtgacttttggacccaaccagtagaagtgcaagaaaatcaagtttatcctattgccaacaatggttcggcaagtacacctttctacacagcgctttgtctgtgggtcggtgcagtcttactttcaagtttggcaacaacagaggttcatctatcagataaagataaaaaacgttattctaaacgtgaacaattctttgctcgaatgggaagtttcgtagccattggacttgctcaagccttgattgtaaccttaggaaactactttggtttaggggtagatgtacgagacccattctacagcgtcctctttgccttactgatatccatcgcctttatgataatggtttatgttctagtcgcgctatttggtaatgttggtaaaggaatagcgattattatccttgtactttccatttctggtggtggtgggaactatccaatccaagtttctggaaaattcttccaagccattaatccttatttaccatttacccacgctgttaatcttttacgagaatcggctggagggatttattggccaaacgcttggtttgcaataattatccttgtggcagtatcaattatatttgttgcaattggggccattctcttcccacactttgaagataaaacgaagaaaatttctgaaatcggaagtaagagtcatttcttccattaa

SEQ ID NO: 3 - protein, 901 aa, Lactococcus lactis
VKSIKNTWKLFALDWKRIFKNPVATFLIVALMIIPSLYAWFNIKALWDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSAILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNSVQITLQSIQEFSKNTSSVISVIDQAIADNQLTDDEKEQINSLITNFTNNIAKQREAIQNIVEYMKQVQESNGNHDLDSTIEQLTNLDSLLSELSTRMSHLNDLVQEGDVTKIRAYLSQINDVVNNISDLINKIDVSEISSTINKALTTLINTITDAKGLLNQAQQIDFDALLNSTSQTVSNAITILEKYQGEMPAIKQEIHDANVMLNGNMTTIVNAINEGANLYKNEFPTLKSKLGTASDFFKNDYAGVRKDLTDTLTMANDKMPDVESALNQANDLIQNDWPEIKSGIQKAAAAIQKGEKEVDLGEIVKLLKLDATKESDFLTQPVEVQENQIYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIVFVAIGAFLFPHFEDKTKKISEIGSKSHFFH

SEQ ID NO: 4 - DNA, 2706 bp, Lactococcus lactis
gtgaaaagtattaaaaatacatggaagttgtttgccctagactggaagcgaatatttaaaaatcccgtggcgacttttttaattgtcgccctaatgataattccctctttatatgcttggtttaatattaaagccctgtgggacccctatgccaatacttcgcaactgccaattgcggtctatagtgcagataaatctgcaagttttcaagataaaaccatcaatattggtgatgaagtactgaaaaatttaaagaaaaatcaccaattaggctggaaatttgttgattccaaagcagatttagataaaggtgtgaaatctggaaaatactatgctggaatctatttaccaaaggatttttcaaaagacctcttaagtttcaccacgggagatattaaaaaaccacaaattgtttattcaattaacgaaaaaatcaatgccatcgctcccaaaataacatcaaaaggagcctcttctctacaatcgcaaatttctgaagagtttatcaaaacggcaagtagtacccttttaaaaaccttcaatacaattggttacgatattgataaaaacatggtaagtattcagaaggttaaatcagcgattttatcaactaatgataatttaggaaccattgataaatacactcaacaagttgttgatcttcatggaaaaatgccagaaattaaagaaaaactggcaaaagctaatgaatttatcacttatttaccagaagtagatgctttaggtcaaaaggtcattaaattaaatgataaaatgccagaaattgataaaagtttttcactcgttttgacccttcaagagaaaatacctgagattcaaaatgctggaaaacaaatctcgatgattgatgatgactttgcgtcagtagaaagtaccatgactcaaggcattcaagaagccaaacaaggtttacaaattatcaatcaagtccaaaaatcaatgccagacattaaaaaattgggacaagatgctgatagtttggggactataaccttggatgcggcgaaaaaaatgcagagtgcgttgcctagcattacaaatagcgttcaaataacacttcaatcaattcaagagttctcaaaaaatacttcatccgttatttctgttattgaccaagcgattgccgataatcaattaactgatgatgaaaaggaacaaataaatagcttaattaccaattttactaataacattgctaaacaaagagaagccattcaaaatattgttgaatatatgaagcaggtgcaagaaagcaatggaaatcacgatttagattctactattgagcaattaactaatctagattcgctcttgtcagaacttagtaccagaatgagtcacctaaatgacttagtgcaagagggtgatgtaactaagattcgagcttatctatcacaaatcaatgatgtagtaaataatatttctgatttaattaataaaattgatgtcagtgagattagtagtacaattaataaagctctaacaacattaattaataccataacagacgctaaaggtttattaaatcaagctcaacaaatcgactttgatgccttactaaattcaacaagccaaacagtttctaatgctattaccattttagaaaaatatcaaggcgaaatgccagccattaaacaagaaattcatgatgcgaatgtgatgttaaatggcaatatgacaactattgttaatgcaatcaatgagggagctaatctctataaaaatgagtttcctactcttaaaagtaagttagggacagcttctgatttctttaaaaatgattatgctggtgttcgtaaagatttgacagataccttaacaatggcaaatgataaaatgcctgatgttgagtctgctttgaatcaagccaatgatttaatccaaaatgattggcctgaaatcaaatcaggaattcaaaaagcagccgcagctattcaaaaaggtgaaaaagaagttgacttaggtgaaatcgtcaaactcctaaaattagatgcgaccaaagagagcgacttcctgacacaaccagttgaagtccaagaaaatcaaatctacccgattgccaacaatggttcagcaagtacacctttctatactgcgctttgtctgtgggtcggtgcagtcttactttcaagtttggcaacaacggaggttcacttatcagataaagataaaaaacgttattctaaacgtgaacaattctttgctcgaatgggaagtttcgtagccattggacttgctcaagccttgattgtaacgcttggaaattattttggtttaggagtagatgtaagagatccattctacagtgttctctttgccttactaatatccatagcctttatgataatggtttatgttctggtcgcactgtttggcaatgtaggtaaaggaatagcgattatcatcctggttctttccatttctggtggtggtgggaactatccaatccaagtttctggaaaattcttccaagccattaatccttatttaccatttactcatgctgtcaaccttttacgagaatcagcgggaggaatttactggccaaatgcttggtttgcgataatcattcttgtagcagtgtcaattgtgtttgttgcaataggtgccttcctcttcccacactttgaagataaaacgaagaaaatttccgagatcggaagcaagagtcatttcttccattaa

SEQ ID NO: 5 - protein, 901 aa, Lactococcus lactis subsp. cremoris
VKSIKNTWKLFALDWKRIFKNPVATFLIVALMIIPSLYAWFNIKALWDPYANTSQLPIAVYSADKPASFQDKTINIGDEVLKNLKKNHQLGWKFVDSKAELDKGVKSGKYYAGIYLPKDFSKDLLSFTTGDIKKPQIVYSINEKINAIAPKITSKGASSLQSQISEEFINTASSTLLKTFNTIGYDIDKNMVSIQKVKSAILSTNDNLGTIDKYTQQVVDLHGKMPEIKEKLAKANEFITYLPEVDSLGQKVISLNDKMPEIDKSLSLVLTLQEKIPEIQNAGKQISMIDEDFASVESTMTQGIQEAKDGLQIINQVQKSMPDIQKLGQDADSLGTVTLDAAKKMQSALPSITNSVQITLQSIQEFSKNTSSVLAVIDQAIADNQLTDEEKEQINSLITNFINNIARQREAIQNIVDYMKQVQESNGSHDLDSTIEQLSNLDSLLSGLSTRMNHLNELVQNGDVSEIRAYLSQINDVVTKISNLVDNIDVSGISKTIDTVLTSLTKTITDAKGLLNQAQQIDFEALLNSTSQTVSNAVTILEKYQGEMPAIKQEIHDANLMLNGNMTTIVNAINEGASLYKNEFPILKTKLGTASDFFKNDYAGVRKDLTDTLTMANEKMPDVESALNQANSLIQNDWPEIKSGIQKAATAIQKGEKEVDLGEIVKLLKLDATKESDFLTQPVEVQENQVYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIIFVAIGAILFPHFEDKTKKISEIGSKSHFFH

SEQ ID NO: 6 - DNA, 2706 bp, Lactococcus lactis subsp. cremoris
gtgaaaagtataaaaaatacctggaagttgtttgccctagactggaaacgaatttttaaaaatcctgtggcgacctttttaatcgttgccttaatgataattccctctttatatgcttggtttaacattaaagccttgtgggacccttatgccaatacctcgcaattgccaattgcagtctacagtgcagataaacctgctagttttcaagataaaaccataaacattggtgatgaagttctgaagaatttaaagaaaaatcatcaattaggttggaaatttgtagattctaaagcagaattagataaaggagttaaatccggaaaatactatgctggaatttacttaccaaaggatttttcaaaagaccttttaagttttacaaccggtgatattaaaaagccacaaatagtttattctattaatgaaaagataaatgctattgctcccaaaataacctctaagggtgcttcatctcttcaatcacagatttctgaagagtttattaatacggcaagtagtacactcttaaaaacctttaatacgattggttacgatattgataaaaatatggtcagcattcaaaaggttaagtcagcaattttatcaacaaacgataatctaggaaccattgacaaatacacccaacaagtggttgaccttcatggtaaaatgccagaaatcaaagaaaaactggctaaggctaacgaatttattacttatttacccgaagttgattctttaggtcaaaaagtaattagtttgaatgataaaatgcctgaaattgataaaagtttgtcacttgttttgactcttcaagaaaaaattcctgaaattcaaaacgctggtaaacaaatttcgatgattgatgaagactttgcgtcagttgaaagtactatgacccaaggaattcaagaagcaaaagacggcttgcaaatcatcaatcaagtccaaaaatcaatgccagacattcaaaaattaggacaagatgcagatagtttagggactgtgacattggatgcagcgaaaaaaatgcagagcgcattgcctagtatcacgaatagcgttcaaatcactctacagtctattcaagaattttcaaaaaatacttcctcagttctcgctgttattgaccaagcaatagctgataatcagctcacagatgaagaaaaagaacaaataaatagtttaattaccaactttattaacaatatcgcccgacaaagagaagcaattcaaaatattgttgattacatgaaacaagtccaagagagcaatggaagtcatgatttagactcaacaattgagcaactcagtaatcttgattctttactgtcaggacttagtactagaatgaatcatctgaatgagttagtacaaaatggtgatgtatctgaaattcgtgcttacttatcccaaattaatgatgtggttactaaaatctctaatttagttgataatattgatgtgagtggaattagtaaaacaattgatacagttttaacaagcttgactaaaacaatcactgatgccaaaggtttattaaatcaagctcaacaaattgactttgaagctttactaaattcaacaagtcaaacagtttctaatgctgttaccattttagaaaaatatcaaggtgaaatgccagccatcaaacaagaaattcatgatgcaaacttgatgttaaatggaaatatgacgacaattgtcaatgcaattaacgaaggggctagtctttataaaaatgagttccctatccttaaaactaagttgggcacagcttctgatttctttaaaaatgattatgctggtgttcgtaaagatttaacagataccttaacaatggcaaatgagaaaatgcctgacgtcgagtctgctttgaatcaagccaatagtttaatccaaaatgactggcctgaaatcaaatcgggcattcaaaaagcagcaacagccattcaaaaaggtgaaaaagaagttgatttaggagagattgttaaacttctaaaacttgatgctactaaagagagtgactttttgacccaaccagtagaagtgcaagaaaatcaagtttatcctattgccaacaatggttcggcaagtacacctttctacacagcgctttgtctgtgggtcggtgcagtcttactttcaagtttggcaacaacagaggttcatctatcagataaagataaaaaacgttattctaaacgtgaacaattctttgctcgaatgggaagtttcgtagccattggacttgctcaagccttgattgtaaccttaggaaactactttggtttaggggtagatgtacgagacccattctacagcgtcctctttgccttactgatatccatcgcctttatgataatggtttatgttctagtcgcgctatttggtaatgttggtaaaggaatagcgattattatccttgtactttccatttctggtggtggtgggaactatccaatccaagtttctggaaaattcttccaagccattaatccttatttaccatttacccacgctgttaatcttttacgagaatcggctggagggatttattggccaaacgcttggtttgcaataattatccttgtggcagtatcaattatatttgttgcaattggggccattctcttcccacactttgaagataaaacgaagaaaatttctgaaatcggaagtaagagtcatttcttccattaa

SEQ ID NO: 7 - protein, 901 aa, Lactococcus lactis subsp. lactis
VKSIKNTWKLFALDWKRIFKNPVATFLIVALMIIPSLYAWFNIKALWDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNSVQITLQSIQEFSKNTSSVISVIDQAIADNQLTDDEKEQINSLITNFTNNIAKQREAIQNIVEYMKQVQESNGNHDLDSTIEQLTNLDSLLSELSTRMSHLNDLVQEGDVTKIRAYLSQINDVVNNISDLINKIDVSEISSTINKALTTLINTITDAKGLLNQAQQIDFDALLNSTSQTVSNAITILEKYQGEMPAIKQEIHDANVMLNGNMTTIVNAINEGANLYKNEFPTLKSKLGTASDFFKNDYAGVRKDLTDTLTMANDKMPDVESALNQANDLIQNDWPEIKSGIQKAAAAIQKGEKEVDLGEIVKLLKLDATKESDFLTQPVEVQENQIYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIVFVAIGAFLFPHFEDKTKKISEIGSKSHFFH

SEQ ID NO: 8 - protein, 856 aa, Lactococcus lactis subsp. lactis
VGDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNSVQITLQSIQEFSKNTSSVISVIDQAIADNQLTDDEKEQINSLITNFTNNIAKQREAIQNIVEYMKQVQESNGNHDLDSTIEQLTNLDSLLSELSTRMSHLNDLVQEGDVTKIRAYLSQINDVVNNISDLINKIDVSEISSTINKALTTLINTITDAKGLLNQAQQIDFDALLNSTSQTVSNAITILEKYQGEMPAIKQEIHDANVMLNGNMTTIVNAINEGANLYKNEFPTLKSKLGTASDFFKNDYAGVRKDLTDTLTMANDKMPDVESALNQANDLIQNDWPEIKSGIQKAAAAIQKGEKEVDLGEIVKLLKLDATKESDFLTQPVEVQENQIYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIVFVAIGAFLFPHFEDKTKKISEIGSKSHFFH

SEQ ID NO: 9 - protein, 244 aa, Lactococcus lactis subsp. lactis
VKSIKNTWKLFALDWKRIFKNPVATFLIVALMIIPSLYAWFNIKALWDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKCQKLKKNWQKLMNLSLIYQK

SEQ ID NO: 10 - protein, 864 aa, Lactococcus lactis subsp. lactis
MLGLILKPCGDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNSVQITLQSIQEFSKNTSSVISVIDQAIADNQLTDDEKEQINSLITNFTNNIAKQREAIQNIVEYMKQVQESNGNHDLDSTIEQLTNLDSLLSELSTRMSHLNDLVQEGDVTKIRAYLSQINDVVNNISDLINKIDVSEISSTINKALTTLINTITDAKGLLNQAQQIDFDALLNSTSQTVSNAITILEKYQGEMPAIKQEIHDANVMLNGNMTTIVNAINEGANLYKNEFPTLKSKLGTASDFFKNDYAGVRKDLTDTLTMANDKMPDVESALNQANDLIQNDWPEIKSGIQKAAAAIQKGEKEVDLGEIVKLLKLDATKESDFLTQPVEVQENQIYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIVFVAIGAFLFPHFEDKTKKISEIGSKSHFFH

SEQ ID NO: 11 - protein, 864 aa, Lactococcus lactis subsp. lactis; identical to SEQ ID NO: 10

SEQ ID NO: 12 - protein, 373 aa, Lactococcus lactis subsp. lactis
VKSIKNTWKLFALDWKRIFKNPVATFLIVALMIIPSLYAWFNIKALWDPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNNTSINSRVLKKYFIRYFCY

SEQ ID NO: 13 - protein, 864 aa, Lactococcus lactis subsp. lactis
MLGLILKPCGTPYANTSQLPIAVYSADKSASFQDKTINIGDEVLKNLKKNHQLGWKFVDSKADLDKGVKSGKYYAGIYLPKDFSKDLLSFTTGDIKKPQIVYSINEKINAIAPKITSKGASSLQSQISEEFIKTASSTLLKTFNTIGYDIDKNMVSIQKVKSVILSTNDNLGTIDKYTQQVVDLHGKMPEIKEKLAKANEFITYLPEVDALGQKVIKLNDKMPEIDKSFSLVLTLQEKIPEIQNAGKQISMIDDDFASVESTMTQGIQEAKQGLQIINQVQKSMPDIKKLGQDADSLGTITLDAAKKMQSALPSITNSVQITLQSIQEFSKNTSSVISVIDQAIADNQLTDDEKEQINSLITNFTNNIAKQREAIQNIVEYMKQVQESNGNHDLDSTIEQLTNLDSLLSELSTRMSHLNDLVQEGDVTKIRAYLSQINDVVNNISDLINKIDVSEISSTINKALTTLINTITDAKGLLNQAQQIDFDALLNSTSQTVSNAITILEKYQGEMPAIKQEIHDANVMLNGNMTTIVNAINEGANLYKNEFPTLKSKLGTASDFFKNDYAGVRKDLTDTLTMANDKMPDVESALNQANDLIQNDWPEIKSGIQKAAAAIQKGEKEVDLGEIVKLLKLDATKESDFLTQPVEVQENQIYPIANNGSASTPFYTALCLWVGAVLLSSLATTEVHLSDKDKKRYSKREQFFARMGSFVAIGLAQALIVTLGNYFGLGVDVRDPFYSVLFALLISIAFMIMVYVLVALFGNVGKGIAIIILVLSISGGGGNYPIQVSGKFFQAINPYLPFTHAVNLLRESAGGIYWPNAWFAIIILVAVSIVFVAIGAFLFPHFEDKTKKISEIGSKSHFFH

(19) United States
(12) Patent Application Publication
(10) Pub. No.: US 2023/0227507 A1
(43) Pub. Date: Jul. 20, 2023

(54) METHOD OF INCREASING NISIN PRODUCTION IN LACTOCOCCUS LACTIS

(71) Applicant: Chr. Hansen A/S, Hoersholm (DK)

(72) Inventors: Thomas Janzen, Hoersholm (DK); Ditte Ellegaard Christiansen, Hoersholm (DK); Mia Vissing Mikkelsen, Hoersholm (DK); Thomas Eckhardt, Hoersholm (DK)

(73) Assignee: Chr. Hansen A/S, Hoersholm (DK)

(21) Appl. No.: 18/008,797

(22) PCT Filed: Jun. 10, 2021
(86) PCT No.: PCT/EP2021/065664

§ 371 Date: Dec. 7, 2022

(30) Foreign Application Priority Data
Jun. 11, 2020 (EP) 20179466.6

(51) Int. Cl. C07K 14/315; A23C 19/032; C12N 1/20

(52) U.S. Cl. CPC C07K 14/315; A23C 19/0323; C12N 1/205; A23Y 2240/41; C12R 2001/46

(57) ABSTRACT

The present invention provides a method of increasing nisin production in nisin-producing Lactococcus lactis by inactivating the phage infection protein Pip. The invention also provides nisin-producing Lactococcus lactis in which the phage infection protein is inactivated. The nisin-producing Lactococcus lactis can be used in starter culture for manufacturing food products or other industrial applications.

### FIELD OF THE INVENTION

The present invention generally relates to the field of bacteriocin, in particular to nisin.

### BACKGROUND OF THE INVENTION

Bacteriocins are low molecular weight antimicrobial peptides produced by bacteria, in particular lactic acid bacteria, that are inhibitory to other bacteria. Nisin is a bacteriocin naturally produced by the dairy starter culture Lactococcus lactis subsp. lactis. It is made by a dairy starter starter microorganism growing in milk and occurs in both soured milk and cheese, albeit at low levels. Nisin possesses a broader antimicrobial spectrum than most other bacteriocins, extending to a wide variety of Gram-positive bacteria, including sporeformers.

Nisin is an approved food additive for use in a broad range of dairy and non-dairy products worldwide, and received Generally Recognized As Safe (GRAS) status in 1988 from the FDA.

Nisin (E 234) is currently an authorized food additive in the European Union under Annex II to Regulation (EC) 1333/2008. The specifications for Nisin (E 234) is assigned EINECS (European INventory of Existing Commercial chemical Substances) number 215-807-5. As a food additive it must be produced by Lactococcus lactis subsp. lactis

Structurally, nisin is a 34 amino acid polypeptide which presents cationic and hydrophobic characteristics. With a molar mass of close to 3500 Da, it contains three unusual amino acids (dehydroalanine, lanthionine and β-methyl-lanthionine) and five internal disulphide bridges. A number of nisin variants have been discovered since the original nisin A was characterized. Nisin variants of lactococcal origin are similar to each other. Nisin A and nisin Z, which differ by a single amino acid substituting histidine at position 27 (in nisin A) and asparagine (in nisin Z). Other variants include nisin F, nisin H, nisin J, nisin U2, nisin U, nisin P, nisin O4 and nisin O123 (Newstead, Logan L., et al. “Staphylococcal-Produced Bacteriocins and Antimicrobial Peptides: Their Potential as Alternative Treatments for Staphylococcus aureus Infections.” Antibiotics 9.2 (2020): 40).

The first demonstration of the utility of nisin in dairy technology was made in 1951 when inhibition of gas-blowing by anaerobic sporeformers in Swiss type cheese was made with a nisin-producing strain of L. lactis subsp. lactis (Hirsch, A. “Growth and nisin production of a strain of Streptococcus lactis.” Microbiology 5.1 (1951): 208-221). A small number of Lactococcus lactis subsp. lactis strains were found to naturally produce nisin. For example, NCDO1402, NCDO1404, CNRZ148, CNRZ150, ATCC11454 were described by Yezzi et al. 1993 (Yezzi, T. L., A. B. Ajao, and E. A. Zottola. “Increased nisin in cheddar-type cheese prepared with pH control of the bulk starter culture system.” Journal of dairy science 76.10 (1993): 2827-2831). Nisin-producing YB23 strain, isolated from raw milk, was reported by Y. Tuncer (Tuncer 2009, Phenotypic and genotypic characterization of nisin-producing Lactococcus lactis subsp. lactis YB23 isolated from raw milk in Turkey, Biotechnology & Biotechnological Equipment, 23:4, 1504-1508). Nisin-producing UL719, isolated from raw milk cheese, was shown to produce nisin Z (Bouksaim et al., 2000, Int. J. Food Microbiol. vol. 59, pp. 141-156). IPLA 729, a nisin Z producer isolated from raw milk cheese, was shown to grow and produce nisin Z in milk (Rilla et al., “Inhibition of Clostridium tyrobutyricum in Vidiago cheese by Lactococcus lactis ssp. lactis IPLA 729, a nisin Z producer.” International journal of food microbiology 85.1-2 (2003): 23-33).

Lactococcal starter cultures that produces nisin in situ during fermentation and acidification of food are commercially available.

Lactococcal starter cultures have several important modes of action in fermented foods, including acidification of the food, slowing down growth of spoilage flora by in situ production of inhibitory molecules such as nisin, and by taking the space and nutrients from the spoilage flora.

There is a need to provide further lactococcal cultures which produce nisin, preferably more nisin than existing commercial products. It is desirable to provide further lactococcal strains with higher inhibitory effect against clostridium. Such strains could be used more efficiently and economically for industrial applications.

Due to the wide commercial use of nisin in the food industry, nisin production by lactococcal strains has been a subject of research in the field.

Zhang et al. showed that an overexpression of hdeAB, ldh and murG makes better performance on the robustness and nisin production of cells (Zhang, Y. F. et al. Genome shuffling of Lactococcus lactis subspecies lactis YF11 for improving nisin Z production and comparative analysis. J Dairy Sci 97, 2528-2541 (2014)).

Cheigh et al. introduced multicopy genes, nisZ, nisRK, or nisFEG in Lactococcus lactis subsp. lactis A164 and observed improved nisin production (Cheigh, C.-I., Park, H., Choi, H.-J. & Pyun, Y.-R. Enhanced nisin production by increasing genes involved in nisin Z biosynthesis in Lactococcus lactis subsp. lactis A164. Biotechnology letters 27, 155-160 (2005)).

There is a general concern over the use of genetically modified organisms (GMOs) in foods and agriculture. Many of the attempts currently known in the art to increase nisin-production make use of recombinant DNA technology. The resulting strains will be considered genetically modified organisms (GMOs) and, as such, will be regulated by the rules and regulations in the countries in which the strains are to be produced or used. The invention makes it possible to use non-GMO methods to improve nisin production, as shown in the examples. As a result, products obtained would not require GMO labelling.

Lactococcus strains with increased nisin production without the use of recombinant DNA technology is highly desirable. Such methods can be used to improve the economics of nisin production and allow its production in heterologous hosts. This serves as an alternative strategy to the manipulation of culture media to increase nisin production, for example as described by Joazala et al. (Jozala et al., “Increase of nisin production by Lactococcus lactis in different media.” African Journal of Biotechnology 4.3 (2005): 262-265). Furthermore, it may improve the value of the strain as a starter culture, as it may provide the inhibition of pathogen and/or spoilage flora through an increased in situ nisin production as well as provides a favorable acidification profile and sensory impacts.

### SUMMARY OF THE INVENTION

The present invention is based in part on the surprising finding that lactococcal strains deficient in phage infection protein (referred to herein also as Pip) produce increased levels of nisin. Based on this, it is now possible to provide strains in which nisin production can be increased. This can be done by mutating the pip gene in the mother strain and select from the mutants whose nisin production is increased.

The phage infection protein (Pip) is a membrane bound protein with some similarities to ABC transporters. The physiological role of the protein in the cell is still unknown. The amino acid sequence of the phage infection protein predicts multiple-membrane-spanning regions, suggesting that it may be anchored to the plasma membrane (Mooney et al., “Subcellular location of phage infection protein (Pip) in Lactococcus lactis.” Canadian Journal of Microbiology 52.7 (2006): 664-672).

Although the physiological role of Pip is unclear, it is known that Pip is required for infection of L. lactis by some bacteriophages. Geller et al. discovered that L. lactis C2 became resistant to bacteriophages of the c2 group (also referred herein as c2 type phage) as a result of inactivation of the lactococcal protein Pip (Geller et al., “Cloning of a chromosomal gene required for phage infection of Lactococcus lactis subsp. lactis C2.” Journal of bacteriology 175.17 (1993): 5510-5519). It was later suggested that lactococcal phage adsorb initially to a cell wall carbohydrate of the host cell, and subsequently to the host cell membrane protein Pip, which leads to ejection of the phage genome (Monteville et al., “Lactococcal bacteriophages require a host cell wall carbohydrate and a plasma membrane protein for adsorption and ejection of DNA.” Appl. Environ. Microbiol. 60.9 (1994): 3204-3211). Babu et al. suggested that Pip may be required for infections by many but not all phages of L. lactis (Babu, K. S., et al., “Characterization of a cloned gene (pip) from Lactococcus lactis required for phage infection.” Developments in biological standardization 85 (1995): 569).

As yet there has been no report on possible relationship between the phage infection protein and nisin production. It is therefore much to the inventors' surprise that mutation in the gene leads to an increase of nisin production.

The phage infection protein had been discovered and known for decades. It is designated according to the transporter classification system given by the Transport Classification Database as TC #3.A.1.155.1. The first Pip was identified by Geller et al. and has the polypeptide sequence as set forth in SEQ ID NO: 1, encoded by the chromosomal polynucleotide as set forth in SEQ ID NO. 2 (Geller et al., “Cloning of a chromosomal gene required for phage infection of Lactococcus lactis subsp. lactis C2.” Journal of Bacteriology 175.17 (1993): 5510-5519). Further, Pip has been identified in other L. lactis strains. For example, Pip can be found in L. lactis IL1403, the first completely sequenced lactococcal strain which was widely used as model microorganism for both fundamental and applied research (Bolotin et al., “The complete genome sequence of the lactic acid bacterium Lactococcus lactis ssp. lactis IL1403.” Genome research 11.5 (2001): 731-753). The Pip polypeptide sequence in the strain MG1363 is as set forth in SEQ ID NO: 3, encoded by the polynucleotide sequence as set forth in SEQ ID NO:4. In addition, Pip is present in the Lactococcus lactis subsp. cremoris strain MG1363, a lactococcal strain most intensively studied throughout the world. The Pip polypeptide sequence of the strain MG1363 is as set forth in SEQ ID NO: 5, encoded by the polynucleotide sequence as set forth in SEQ ID NO:6 (Wegmann et al., “Complete genome sequence of the prototype lactic acid bacterium Lactococcus lactis subsp. cremoris MG1363.” Journal of Bacteriology 189.8 (2007): 3256-3270)). The pip sequence shares high homology between L. lactis strains. It is well within the skill of an ordinary person in the art to identify the presence of pip in a lactococcal strain.

Mutation of pip has been disclosed previously. Roces 2012 reported two L. lactis mutants, D1 and D1-20, derived from L. lactis MG1614, which have deletion of the pip gene. The absence of a functional Pip explains their resistance towards c2 type phages (Roces, Clara, et al., “Isolation of Lactococcus lactis mutants simultaneously resistant to the cell wall-active bacteriocin Lcn972, lysozyme, nisin, and bacteriophage c2.” Appl. Environ. Microbiol. 78.12 (2012): 4157-4163). It should be noted that L. lactis MG1614 is known to be nisin-negative (i.e. does not produce nisin) (Dodd, Helen M., et al., “Physical and genetic analysis of the chromosomally located transposon Tn5301, responsible for nisin biosynthesis.” Nisin and novel lantibiotics. Escom Publishers, Leiden, The Netherlands (1991): 231-242). Although Roces et al. obtained lactococcal pip mutants, clearly these mutants would not produce nisin.

There is no hint in Roces 2012 that pip gene is linked to nisin production, or pip mutation would lead to increased nisin production by the lactococcal strain.

Based on the present finding, the inventors provide a strategy to increase nisin production of a nisin-producing Lactococcus lactis mother strain, including strains belong to the subspecies of Lactococcus lactis subsp. lactis, Lactococcus lactis subsp. cremoris or Lactococcus lactis subsp. lactis biovar diacetylactis. The mother strain may produce different variants of nisin, for example, nisin A or nisin Z. The term “nisin-producing” means that the strain is able to express nisin when grown in a condition which allows for nisin production.

Nisin increase can be achieved by preparing mutants in which the phage infection protein is inactivated, and selecting from the mutants daughter strains in which nisin production is increased compared to the mother strain. Nisin production can be measured by growing the strain in a condition which allows for nisin production. In one preferred embodiment, nisin production is evaluated according to the culturing conditions as described in Example 2.

The present invention provides a method which comprises mutating the Pip encoding gene or its regulatory sequences, such as by substitution, truncation, deletion, point mutation, and/or knock-out. Mutation with genetically modified techniques as well as non-genetically modified techniques are included in the present application. Genetically modified techniques offer a straight-forward modification, whereas non-genetically modified strategies are preferred if regional rules or market demands require so.

The present invention also provides a method of obtaining nisin by culturing a nisin-producing Lactococcus lactis in which the phage infection protein is inactivated, in a condition which allows for nisin-production.

In one aspect, the invention provides a method of increasing nisin production in nisin-producing Lactococcus lactis, comprising:
providing one or more nisin-producing Lactococcus lactis strains which express the phage infection protein (TC #3.A.1.155.1) as the mother strain,
obtaining one or more Lactococcus lactis mutants in which the phage infection protein is inactivated, and
selecting from the obtained mutants one or more Lactococcus lactis strains whose nisin production is increased compared to the mother strain.

In one aspect, the present invention provides a method of increasing nisin production in nisin-producing Lactococcus lactis, comprising:
providing one or more nisin-producing Lactococcus lactis strains which express the phage infection protein (TC #3.A.1.155.1) as mother strain,
mutating of the mother strain gene(s) which encode the phage infection protein in the mother strain or regulate the expression of genes which encode the phage infection protein, for example by substitution, truncation, deletion, point mutation and/or knock-out,
obtaining one or more Lactococcus lactis mutants in which the phage infection protein is inactivated, and
selecting from the obtained mutants one or more Lactococcus lactis daughter strains whose nisin production is increased compared to the mother strain.

It is within the scope of the present application that mutations may also be introduced in the sequences which regulates the expression of the phage infection protein.

In another aspect, the present method comprises:
providing one or more nisin-producing Lactococcus lactis strains which express the phage infection protein (TC #3.A.1.155.1) as mother strain,
mutating of the mother strain gene(s) which encode the phage infection protein or regulate the expression thereof by deleting fully or partially the gene(s),
obtaining one or more Lactococcus lactis mutants in which the phage infection protein is inactivated, and
selecting from the obtained mutants one or more Lactococcus lactis daughter strains whose nisin production is increased compared to the mother strain.

In a further aspect, the present method comprises:
providing one or more nisin-producing Lactococcus lactis strains which express the phage infection protein (TC #3.A.1.155.1) as mother strain,
phage-hardening the mother strain against one or more bacteriophages which recognize the phage infection protein, for example a c2 type phage,
obtaining one or more Lactococcus lactis phage resistant mutants in which the phage infection protein is inactivated, and
selecting from the obtained mutants one or more Lactococcus lactis daughter strains whose nisin production is increased compared to the mother strain.

In one aspect, the present method comprises:
providing one or more nisin-producing Lactococcus lactis strains which express the phage infection protein (TC #3.A.1.155.1) as mother strain,
deleting fully or partially the gene(s) which encode the phage infection protein and obtaining one or more Lactococcus lactis mutants in which the phage infection protein is inactivated, and
selecting from the obtained mutants one or more Lactococcus lactis daughter strains whose nisin production is increased compared to the mother strain.

A further aspect of the invention provides one or more daughter strains which can be obtained by the present method as disclosed herein. The daughter strains are able to produce more nisin than the mother strains when grown under the same condition which allows for nisin production. In one preferred embodiment, nisin production is evaluated according to the culturing conditions and methods as described in Example 2.

The present invention provides a nisin-producing Lactococcus lactis strain with inactivated phage infection protein (TC #3.A.1.155.1). An example includes the Lactococcus lactis strain deposited as DSM 33302. The inventors demonstrate in the present application that the strain has increased nisin production compared to the mother strain it was derived from.

Included herein are also nisin-producing Lactococcus lactis strains obtained or obtainable by phage-hardening against a bacteriophage which recognizes the phage infection protein (TC #3.A.1.155.1).

In a further aspect the present invention provides a nisin-producing Lactococcus lactis strain comprising a phage infection protein with the polypeptide sequence as set forth in SEQ ID NO: 8, SEQ ID NO: 9, SEQ ID NO: 10 or SEQ ID NO: 11.

Furthermore, the present invention provides a Lactococcus lactis strain deposited as DSM 33302.

The present invention also provides a composition, such as a starter culture composition, which comprises a nisin-producing Lactococcus lactis strain that is obtained or obtainable according to the presently disclosed methods. Included herein are compositions comprising nisin-producing Lactococcus lactis strain(s) with inactivated phage infection protein (TC #3.A.1.155.1), preferably with those as set forth in SEQ ID NO: 8, SEQ ID NO: 9, SEQ ID NO: 10 or SEQ ID NO: 11 or variants thereof.

Food products comprising such strains are also part of the present invention.

Furthermore, the present invention provides a method of obtaining nisin from the daughter strains disclosed herein. The method comprises growing, under a condition which allows for nisin production, a nisin-producing Lactococcus lactis strain that is obtained or obtainable according to the presently disclosed methods, and purifying therefrom nisin. Such conditions are exemplified by the present application or can be readily determined by a skilled person in the art.

### BRIEF DESCRIPTION OF THE FIGURES

FIG. 1 depicts the alignment of Pip polypeptide sequences of DSM 18874 (SEQ ID NO: 7) and four Pip mutants A, B, C and D (SEQ ID NO: 8-11) obtained using the present invention.

FIG. 2 shows nisin levels for strain A and DSM 18874 when fermenting as single strain or together with cheese starter culture C501.

FIG. 3 Acidification profiles of DSM 18874 in milk with and without the addition of phage.

FIG. 4 Acidification profiles of strain A in milk with and without the addition of phage.

FIG. 5 Acidification profiles of strain B in milk with and without the addition of phage.

FIG. 6 Acidification profiles of strain C in milk with and without the addition of phage.

FIG. 7 Acidification profiles of strain D in milk with and without the addition of phage.

FIG. 8 depicts the alignment of Pip polypeptide sequences of Pip reported by Geller (SEQ ID NO: 1) and four Pip mutants A, B, C and D (SEQ ID NO: 8-11) obtained in the present invention. The predicted transmembrane domains at positions 14-38, 694-714, 738-760, 768-790, 796-819 or 849-873 are marked bold.

FIG. 9 depicts the alignment of Pip polypeptide sequences of IL1403 Pip (SEQ ID NO: 3) and six Pip mutants A, B, C, D, E and F (SEQ ID NO: 8-13) obtained in the present invention.

### BRIEF DESCRIPTION OF THE SEQUENCES

SEQ ID NO: 1 polypeptide sequence of Pip reported by Geller 1993

SEQ ID NO: 2 polynucleotide sequence of Pip reported by Geller 1993

SEQ ID NO: 3 polypeptide sequence of IL1403 Pip

SEQ ID NO: 4 polynucleotide sequence of IL1403 Pip

SEQ ID NO: 5 polypeptide sequence of MG1363 Pip

SEQ ID NO: 6 polynucleotide sequence of MG1363 Pip

SEQ ID NO: 7 polypeptide sequence of DSM 18874 Pip

SEQ ID NO: 8 polypeptide sequence of strain A (DSM 33302) Pip

SEQ ID NO: 9 polypeptide sequence of strain B Pip

SEQ ID NO: 10 polypeptide sequence of strain C Pip

SEQ ID NO: 11 polypeptide sequence of strain D Pip

SEQ ID NO: 12 polypeptide sequence of strain E Pip

SEQ ID NO: 13 polypeptide sequence of strain F Pip

### DETAILED DESCRIPTION OF THE INVENTION

This invention relates to Lactococcus lactis bacteria, referred to here also as Lactococcus lactis strains, which produce nisin. The present inventors surprisingly discovered that Lactococcus lactis, when subjected to mutation in phage infection protein (Pip), exhibited an increase in nisin production. The finding therefore provides a novel strategy to increase nisin production in lactococcal strains.

The present invention discloses a method of increasing existing nisin production in Lactococcus lactis. Nisin belongs to the Class I bacteriocins and is an effective bactericidal agent against Gram-positive bacteria including strains of Lactococcus, Streptococcus, Staphylococcus, Micrococcus, Pediococcus, Lactobacillus, Listeria, Clostridium and Mycobacterium.

In Europe, the safety of nisin (E 234) as a food additive has been evaluated in 2006 by the European Food Safety Authority's Panel on Food Additives, Flavourings, Processing Aids and Materials in Contact with Food.

Nisin forms pores at the cytoplasmatic membrane of the target bacteria to disrupt the proton motive force and the pH equilibrium. This causes leakage of ions and hydrolysis of ATP and results in cell death. Other studies have shown that nisin also interferes with cell wall biosynthesis, mediated by the ability of nisin to bind lipid II, a peptidoglycan precursor in the synthesis of the cell wall of bacteria.

In accordance with the present invention, a nisin-producing Lactococcus lactis strain is selected as the mother strain. Lactococcus lactis (L. lactis) is a well-characterized, food-grade lactic acid bacterium (LAB) with generally recognized as safe (GRAS) status. The first bacterial pure culture of Lactococcus lactis (previously known as Bacterium lactis or Streptococcus lactis) was isolated from boiled milk in 1873. Subsequently, L. lactis became an important starter culture in the food industry, particularly for the production of cheese. L. lactis is described as a non-pathogenic, mesophilic, coccus bacterium of about 0.5 to 1 μm diameter.

A skilled person is able to determine whether a Lactococcus lactis produces nisin. Genes required for nisin synthesis are well known in the art. Nisin is ribosomally synthesized as a precursor peptide that undergoes post-translational modifications, i.e. dehydration of serine and threonine residues and formation of five intramolecular thioether ring structures called (p-methyl) lanthionine residues. The biosynthesis of bacteriocins by a number of lactic acid bacteria is generally encoded by gene clusters containing conserved genes. The complex biosynthesis of nisin is encoded by the chromosomally-located gene cluster nisA(Z)BTCIPRKFEG. The cluster is required for nisin biosynthesis, development of immunity, and regulation of gene expression. Of these genes, the nisA(Z) gene encodes nisin A(Z) precursor peptide consisting of 57-amino acid residues, containing a 23-amino acid residues, N-terminal leader peptide that is involved in directing the modification and targeting process of nisin precursor. NisB and nisC encode membrane-associated proteins involved in the intracellular post-translational modification reaction. The ribosomally synthesized nisin precursor is post-translationally modified such that serine and threonine residues are dehydrated to become dehydroalanine and dehydrobutyrine. Subsequently, five of the dehydrated residues are coupled to upstream cysteines, thus form the thioether bonds that produce the characteristic (b-methyl)lanthionine rings. nisT encodes a putative transporter protein of ABC translocator family that is involved in the translocation of the fully modified nisin precursor across the cytoplasmic membrane. NisP encodes a subtilisin-like protease involved in extracellular proteolytic activation. During or shortly after translocation of the nisin precursor, the leader peptide is removed by the subtilisin-like protease to form an extracellular mature nisin peptide. NisI encodes a lipoprotein involved in the self-protection of the producing bacterium against nisin and nisFEG encodes a putative ABC exporter involved in nisin extrusion. NisR and nisK encode a response regulator and a sensor kinase of the histidine protein kinase family, respectively, that belong to a class of two-component regulatory systems (Cheigh, Chan-Ick, and Yu-Ryang Pyun. “Nisin biosynthesis and its properties.” Biotechnology letters 27.21 (2005): 1641-1648).

Nisin-producing Lactococcus lactis are known in the field. Nisin-producing strains can also be isolated from natural sources such as fermented food. This has been done for example as described in Beasley et al., “Nisin-producing Lactococcus lactis strains isolated from human milk.” Appl. Environ. Microbiol. 70.8 (2004): 5051-5053; Noonpakdee, W., et al., “Isolation of nisin-producing Lactococcus lactis WNC 20 strain from nham, a traditional Thai fermented sausage.” International Journal of Food Microbiology 81.2 (2003): 137-145; and Rodriguez, J. M., et al., “Isolation of nisin-producing Lactococcus lactis strains from dry fermented sausages.” Journal of Applied Bacteriology 78.2 (1995): 109-115.

It is well within the capability of a person skilled in the art to provide Lactococcus lactis which produces nisin as mother strain. For example, one can examine whether a strain harbors nisin gene cluster and/or check for nisin production using for instance agar diffusion bioassay (Pongtharangkul et al., “Evaluation of agar diffusion bioassay for nisin quantification.” Applied microbiology and biotechnology 65.3 (2004): 268-272). It is also possible to isolate and identify nisin-producing strains using the Nisin-Controlled gene Expression (NICE) vector system (Hu et al., “Identification of nisin-producing strains by nisin-controlled gene expression system.” Current microbiology 58.6 (2009): 604-608)).

The mother stain according to the present invention comprises the phage infection protein Pip (TC 3.A.1.155.1). It should be understood that the phage infection protein of the mother strain is functionally active.

According to the present invention regarding the phage infection protein, the term “functionally active” means that the bacterium can be infected by a bacteriophage which recognizes the phage infection protein, or that the bacterium has a pip sequence which is identical or very similar to a pip from a bacterium which can be infected by a bacteriophage which recognizes the phage infection protein. As used herein, “very similar” means at least 90%, such as at least 91%, 92%, 93%, 94%, 95%, 96%, 97%, 98%, 99% or 100% sequence identity.

In contrast, a phage infection protein which is not functionally active is referred to as “functionally inactivated” or simply “inactivated.”

Recognition of c2 type phage is known and described by Millen et al., “Genetic determinants of lactococcal C2 viruses for host infection and their role in phage evolution.” The Journal of general virology 97.8 (2016): 1998).

It is routine work for the skilled person to determine whether the phage infection protein is inactivated. One can for example compare the pip sequence with known pip sequences. Alternatively, one can also grow the bacterium in the presence of a collection of bacteriophages classified as belonging to the c2 type phage and see if infection takes place. However, since the lytic development of bacteriophages involves many mechanisms (such as adsorption of the phages to the host cell surface, injection of phage DNA into the cell, synthesis of phage proteins, replication of phage DNA, assembly of progeny phages and release of progeny from the host), interference with any of these cell-mediated mechanisms may prevent a phage infection. Therefore, one would readily understand that sequence analysis should be made to confirm that the insensitivity to the bacteriophage is due to the mutation of pip.

Thus, an alternative way to measure the inactivity of the protein is to analyze the pip gene sequence to see if it comprises a modification that cause inactivation of the protein. As explained above a mutation may be many things such as a stop codon, an insertion that e.g. cause frame shift, a deletion, a mutation etc. It is routine for a skilled person (e.g. by sequencing the gene) to identify if the gene comprises such a suitable modification.

A skilled person in the art is able to apply c2 type phages useful for the infection of the mother strain and the inactivation of the phage infection protein. Such phages are known from the scientific literature or may be obtained by request to Chr. Hansen A/S, Denmark.

In one embodiment, phage infection protein of the mother strain is preferably a wild-type protein. The term “wild-type” means that the protein comprises an amino acid sequence identical to one which was found in nature.

For example, the phage infection protein of the mother strain may comprise a polypeptide having at least 90%, such as at least 91%, 92%, 93%, 94%, 95%, 96%, 97%, 98%, 99% or 100% sequence identity with the sequence of SEQ ID NO: 1.

The phage infection protein of the mother strain may also comprise a polypeptide having at least 90%, such as at least 91%, 92%, 93%, 94%, 95%, 96%, 97%, 98%, 99% or 100% sequence identity with the sequence of SEQ ID NO: 3.

The phage infection protein of the mother strain may also comprise a polypeptide having at least 90%, such as at least 91%, 92%, 93%, 94%, 95%, 96%, 97%, 98%, 99% or 100% sequence identity with the sequence of SEQ ID NO: 5.

The phage infection protein of the mother strain may also comprise a polypeptide having at least 90%, such as at least 91%, 92%, 93%, 94%, 95%, 96%, 97%, 98%, 99% or 100% sequence identity with the sequence of SEQ ID NO: 7.

For purposes of the present invention, the degree of “sequence identity” between two polypeptide sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al. 2000, Trends Genet. 16: 276-277). One may use the EMBOSS Matcher alignment as described in Madeira, Fábio, et al., “The EMBL-EBI search and sequence analysis tools APIs in 2019.” Nucleic acids research 47.W1 (2019): W636-W641. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

The phage infection protein belongs to the ATP-binding cassette (abc) superfamily. The protein Pip is designated as TC #3.A.1.155.1 in the transporter classification system given by the Transport Classification Database (TCBD) (M. Saier; U of CA, San Diego, Saier M H, Reddy V S, Tamang D G, Vastermark A. (2014)). The TC system is a classification system for transport proteins which is analogous to the Enzyme Commission (EC) system for classification of enzymes. The transporter classification (TC) system is an approved system of nomenclature for transport protein classification by the International Union of Biochemistry and Molecular Biology. TCDB is freely accessible at http://www.tcdb.org which provides several different methods for accessing the data, including step-by-step access to hierarchical classification, direct search by sequence or TC number and full-text searching.

According to TCDB, the Pip family includes large proteins with one N-terminal hydrophobic transmembrane segment, a hydrophilic domain of variable length, and five C-terminal putative transmembrane segments. The lactococcal Pip protein was first described in L. lactis C2 (GenBank accession number L14679). It is a 901 aa membranespanning protein encoded by a 2706-bp gene (Geller et al., 1993) (SEQ ID NO:1; UniProt accession number:.P49022) with homologues in most Gram-positive bacteria.

Based on the pip related sequence information known in art and disclosed herein, a skilled person is able to determine if a gene in another L. lactis technically seen would be a gene which encodes pip. The presence of pip appears to be universally in lactococcal genomes. A skilled person will readily recognize that there are genetic variations in the phage infection protein sequence. By sequence comparison, one is able to determine whether a particular strain comprises genes which encode a Pip protein.

The mother strain according to the present invention comprises a phage infection protein designated as TC #3.A.1.155.1. In one embodiment, the mother strain comprises a phage infection protein encoded by SEQ ID NO:2, SEQ ID NO:4 or SEQ ID NO:6 or by a polynucleotide sequence having at least 55%, such as at least 60%, 65%, 70%, 75%, 80%, 85%, 90%, 95%, 96%, 97%, 98%, 99% or 100% sequence identity with the sequences of any one of SEQ ID NO:2, SEQ ID NO:4 or SEQ ID NO:6.

One of the various advantages of the present invention is that an increase in nisin can be achieved without apparent changes in acidification profile compared to mother strain.

From the nisin-producing Lactococcus lactis strains which comprises the phage infection protein (TC #3.A.1.155.1) as the mother strain, the next step is to obtain one or more Lactococcus lactis mutants in which the phage infection protein is inactivated.

In the present context, the term “mutant” should be understood as a strain derived, or a strain which can be derived, from a strain of the invention (or the mother strain) by means of e.g. genetic engineering, radiation, chemical treatment and/or phage hardening.

It is preferred that the mutant is a functionally equivalent mutant, e.g. a mutant that has substantially the same, or improved, properties (e.g. regarding texture, shear stress, viscosity, gel stiffness, mouth coating, flavor, post acidification, acidification speed, and/or phage robustness) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been exchanged with another nucleotide, or deleted, compared to the mother strain.

Inactivation of pip can be carried out by various means. The protein may be inactivated by suitable modification introduced into the pip gene, including but not limited to an insertion that e.g. causes frame shift, a stop codon, deletion, substitution. It is within the scope of the present application that the mutation would also include mutation in the regulatory sequences which control the expression of the phage infection protein. Such mutations will lead to a decreased or lack of expression of the pip gene. In preferred embodiments, the mutation is made in the promoter or the ribosomal binding site.

As explained below it is routine work for the skilled person to mutate the gene to render the phage infection protein inactivated. For instance by introducing a stop codon or a frameshift insertion in the pip gene, which could give a non-functional gene that would e.g. either express no phage infection protein or express a partial length inactive phage infection protein.

Inactivation can also be done by phage hardening, i.e. rendering the mother strain insensitive for phage infection, where possible. The phage infection protein Pip is reported to be the receptor for c2 type phages and is required for phage infection (Valyasevi et al., “A membrane protein is required for bacteriophage c2 infection of Lactococcus lactis subsp. lactis C2.” Journal of Bacteriology 173.19 (1991): 6095-6100).

One may subject the mother strain to c2 type phage and select for phage resistant mutants in which Pip is inactivated, using methods familiar to those skilled in the art. Phage hardening typically involves exposing the resulting lactic bacterial strain to a bacteriophage which is able to lyse the mother strain, incubating the exposed bacterial cells in a growth medium; and isolating a mutant strain of the mother strain, which mutant strain is not lysed by the bacteriophage.

Phage resistance can be evaluated by use of a standard plaque assay based on the agar overlay method. The plaque assay evaluates the phage resistance of a strain of interest as the difference in pfu/ml (plaque forming units per ml) obtainable with a given bacteriophage on the strain of interest, compared to the pfu/ml obtainable with the same bacteriophage on the mother strain.

In some instances, one will readily understand that the mother strain, although comprising Pip, could still be insensitive to c2 type phage attacks, because the mother strain lacks other features required for phage infection. In this case, other approaches such as DNA recombinant technology could be used.

Methods of inactivating the pip gene are known and well practiced in the dairy industry, since Lactococcus lactis are used as starter cultures for commercial dairy fermentations. It is known that they are susceptible to attacks by lytic bacteriophages, in particular from the phages belonging to the P335, 936 and c2 groups, which are present in the fermentation process and are a major cause of fermentation failure and product defects.

Kraus et al. described the construction of a number of commercially relevant Lactococcus lactis strains where the Pip was inactivated (pip⁻ strains). The pip⁻ strains were completely resistant to prolate bacteriophage of the c2 species but were fully sensitive to other phages (Kraus et al., “Membrane receptor for prolate phages is not required for infection of Lactococcus lactis by small or large isometric phages.” Journal of dairy science 81.9 (1998): 2329-2335). Millen et al. constructed a phage insensitive mutant which was found to have 487-bp deletion in pip gene. This deletion resulted in a Pip which is non-functional as a phage receptor (Millen, Anne M., and Dennis A. Romero. “Genetic determinants of lactococcal C2 viruses for host infection and their role in phage EVOLUTION.” THE JOURNAL OF GENERAL VIROLOGY 97.8 (2016): 1998).

As used herein, the term “bacteriophage” has its conventional meaning as understood in the art i.e. a virus that selectively infects one or more bacteria. Many bacteriophages are specific to a particular genus or species or strain of bacteria. The term “bacteriophage” is synonymous with the term “phage.”

Other routine methods to introduce mutation is by homologous recombination of a suitable DNA fragment into the pip genomic gene sequence (e.g. by use of the publicly available pGhost vectors or by other cloning vectors). The introduced fragment may contain for instance a nonsense (stop) codon, a frameshift mutation, a deletion, a mutation or an insertion.

An example is described by Garbutt et al. (Garbutt, K. C., J. Kraus, and B. L. Geller. “Bacteriophage resistance in Lactococcus lactis engineered by replacement of a gene for a bacteriophage receptor.” Journal of dairy science 80.8 (1997): 1512-1519).

In one preferred embodiment, the mutation includes a N-terminal deletion or a C-terminal deletion. Such deletion may result in a protein lacking fully or partially one or more of the predicted transmembrane domains, including the N-terminal hydrophobic transmembrane segments and C-terminal putative transmembrane segments.

Thus one embodiment of the present invention is a nisin-producing L. lactis strain, preferably Lactococcus lactis subsp. lactis or Lactococcus lactis subsp. cremoris or Lactococcus lactis subsp. lactis biovar diacetylactis, comprising a phage infection protein that lacks fully or substantially one, two, three, four, five or six of the predicted transmembrane domains corresponding to amino acids 14-38, 694-714, 738-760, 768-790, 796-819 or 849-873 of Pip reported by Geller et al. 1993 (SEQ ID NO: 1) (TC #3.A.1.155.1). The term “substantially” refers to a great degree, such as a degree of greater than 75%, such as 80%, 95%, 95% or 98%.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 14-38 of SEQ ID NO: 1, for example the preferred embodiment strain deposited as DSM 33302.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 694-714 of SEQ ID NO: 1.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 738-760 of SEQ ID NO: 1.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 768-790 of SEQ ID NO: 1.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 796-819 of SEQ ID NO: 1.

In one embodiment, the Lactococcus lactis, preferably Lactococcus lactis subsp. lactis or Lactococcus lactis subsp. cremoris or Lactococcus lactis subsp. lactis biovar diacetylactis, lacks fully or substantially the predicted transmembrane domain corresponding to amino acids 849-873 of SEQ ID NO: 1.

It is routine work for the skilled person to choose an adequate strategy to e.g. introduce a suitable modification of the pip gene to inactivate the phage infection protein. Alternatively, one may randomly mutagenize (e.g. by UV radiation) and select for mutations wherein the phage infection protein is inactivated. Further, one could select for relevant spontaneous mutations, wherein the phage infection protein is inactivated.

In some embodiments the method comprises selecting from the mutant strains daughter strains whose nisin production is increased compared to the mother strain. Comparison is made under the same condition which allows for nisin production. It is routine work to grow L. lactis under conditions which allows for nisin production. Such conditions are known and described in the art, including de Arauz, Luciana Juncioni, et al., “Nisin biotechnological production and application: a review.” Trends in Food Science & Technology 20.3-4 (2009): 146-154; De Vuyst, L. “Nutritional factors affecting nisin production by Lactococcus lactis subsp. lactis NIZO 22186 in a synthetic medium.” Journal of Applied Bacteriology 78.1 (1995): 28-33; Jozala, Angela Faustino, et al., “Increase of nisin production by Lactococcus lactis in different media.” African Journal of Biotechnology 4.3 (2005): 262-265; Liu, Xia, et al., “Continuous nisin production in laboratory media and whey permeate by immobilized Lactococcus lactis.” Process Biochemistry 40.1 (2005): 13-24; Kim, W. S., R. J. Hall, and N. W. Dunn. “The effect of nisin concentration and nutrient depletion on nisin production of Lactococcus lactis.” Applied Microbiology and Biotechnology 48.4 (1997): 449-453.

Nisin production can be observed using agar diffusion bioassay known in the art, the most widely used method for quantifying nisin activity (Pongtharangkul et al. 2004). In this method, nisin is allowed to diffuse through agar gel seeded with nisin-sensitive indicator bacteria. The diameter of the inhibition zone produced by growth inhibition of nisin-sensitive indicator bacteria in the agar plate is correlated with the concentration of nisin. Greater nisin concentrations result in larger inhibition zone. A skilled person is aware that factors such as nisin structure, concentration of agar, pH, detergent, number of indicator cells and temperature can affect diffusion of nisin through agar, and would be able to choose a diffusion assay suitable for the selection of strains that have higher nisin product compared to the mother strain.

Nisin production may also be measured, for example by ELISA methods and bioassays using bioluminescence and green fluorescent protein. Alternatively, gas chromatography, high performance liquid chromatography (HPLC), liquid chromatography-mass spectrometry (LC-MS), and liquid chromatography-tandem mass spectrometry (LC-MS/MS), or high performance liquid chromatography-tandem mass spectrometry can also be used.

Natural variants of nisin occur in Lactococcus strains from different isolation sources. Nisin A and Nisin Z are the most common ones, found in many dairy isolates. These two nisin variants share the same structure except for an amino acid at position 27. The following nisin variants are also known and described: nisin Q, nisin U, nisin U2, nisin P, nisin F and nisin H (O'Connor et al., “Nisin H is a new nisin variant produced by the gut-derived strain Streptococcus hyointestinalis DPC6484.” Appl. Environ. Microbiol. 81.12 (2015): 3953-3960.). Nisin Q has four amino acid substitutions when comparing to nisin A at the C-terminal part of the molecule. Antimicrobial activity assays reveal only small differences between the three nisin variants against different target organisms (Yoneyama et al., “Biosynthetic characterization and biochemical features of the third natural nisin variant, nisin Q, produced by Lactococcus lactis 61-14.” Journal of applied microbiology 105.6 (2008): 1982-1990). The present invention is not limited to particular nisin variants. Preferably, nisin produced by the lactococcal strain of the present invention is nisin A or nisin Z.

Nisin measurement can be carried out by a variety of analytical methods known to a skilled person in the art. Agar diffusion techniques are the most widely used. However, limitations include low sensitivity due to interfering substances in food extracts, long microbial culture times, as well as the formation of false inhibitory zones related to the low pH of samples. The ELISA methods and bioassays using bioluminescence and green fluorescent protein are more sensitive and rapid. In recent years high performance liquid chromatography (HPLC), liquid chromatography-mass spectrometry (LC-MS), and liquid chromatography-tandem mass spectrometry (LC-MS/MS), or high performance liquid chromatography-tandem mass spectrometry are used to measure bacteriocin. They allow accurate molecular mass determination of target molecules in crude samples. Such methods are described for example in Mehlis et al., “Liquid chromatography/mass spectrometry of peptides of biological samples.” Analytica Chimica Acta 352.1-3 (1997): 71-83; Zendo et al., “Bacteriocin detection by liquid chromatography/mass spectrometry for rapid identification.” Journal of applied microbiology 104.2 (2008): 499-507; Schneider et al., “Analysis of nisin A, nisin Z and their degradation products by LCMS/MS.” Food chemistry 127.2 (2011): 847-854; Fuselli, Fabio, et al., “Multi-detection of preservatives in cheeses by liquid chromatography-tandem mass spectrometry.” Journal of Chromatography B 906 (2012): 9-18.

In accordance with the present invention, a daughter strain having increased nisin production compared to the mother strain is provided. In one preferred embodiment, nisin production is evaluated according to the culturing conditions and methods as described in Example 2.

With the methods described herein, it is possible to provide a nisin-producing Lactococcus lactis in which pip is mutated. Such strain has a higher nisin-production compared to the mother strain. The present invention includes nisin-producing Lactococcus lactis strains obtained or obtainable by the presently disclosed methods.

In accordance with the present invention, the phage infection protein is mutated, for example due to a frameshift or a stop codon sequence encoding the phage infection protein. Provided herein are nisin-producing Lactococcus lactis strains comprising an inactivated phage infection protein (TC #3.A.1.155.1), preferably Lactococcus lactis subsp. lactis or Lactococcus lactis subsp. cremoris or Lactococcus lactis subsp. lactis biovar diacetylactis.

The phage infection protein can be mutated in one or more of its transmembrane domains.

In one embodiment, the present invention provides nisin-producing Lactococcus lactis strains comprising an inactivated phage infection protein (TC #3.A.1.155.1), preferably Lactococcus lactis subsp. lactis or Lactococcus lactis subsp. cremoris or Lactococcus lactis subsp. lactis biovar diacetylactis, wherein the inactivation is due to a presence of a stop codon in the pip gene. The stop codon leads to the early termination of pip translation.

Preferably, the stop codon is present between the first and second predicted transmembrane domains. The first transmembrane domain corresponds to amino acids 14-38, and the second transmembrane domain corresponds to 694-714 of Pip as set forth in SEQ ID NO: 1 (pip reported by Geller 1993). For example, the phage infection protein may lack fully or substantially one, two, three, four, five or six of the predicted transmembrane domains. The domains correspond to amino acids 14-38, 694-714, 738-760, 768-790, 796-819 or 849-873 of Pip as set forth in SEQ ID NO: 1 (pip reported by Geller 1993).

In accordance with the present invention, provided is a nisin-producing Lactococcus lactis subsp. lactis or Lactococcus lactis subsp. cremoris or Lactococcus lactis subsp. lactis biovar diacetylactis comprising a phage infection protein which lacks fully or substantially one, two, three, four, five or six of the predicted transmembrane domains.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 694-714, 738-760, 768-790, 796-819 or 849-873 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 14-38, 738-760, 768-790, 796-819 or 849-873 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 14-38, 694-714, 768-790, 796-819 or 849-873 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 14-38, 694-714, 738-760, 796-819 or 849-873 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 14-38, 694-714, 738-760, 768-790 or 849-873 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four or five of the predicted transmembrane domains corresponding to amino acids 14-38, 694-714, 738-760, 768-790 or 796-819 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially the predicted transmembrane domains corresponding to amino acids 796-819 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially the predicted transmembrane domains corresponding to amino acids 768-790 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially the predicted transmembrane domains corresponding to amino acids 738-760 of Pip as set forth in SEQ ID NO: 1.

Included herein are also a nisin-producing L. lactis strain, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially the predicted transmembrane domains corresponding to amino acids 694-714 of Pip as set forth in SEQ ID NO: 1.

Daughter strains of the present application are preferably obtained by genetic engineering of phage-hardening a mother strain disclosed herein against a bacteriophage which recognizes the phage infection protein (TC #3.A.1.155.1), for example a bacteriophage belonging to the c2 type phage. The mother strain can be one or more nisin-producing Lactococcus lactis strains which express the phage infection protein (TC #3.A.1.155.1).

Included in the present application is a nisin-producing Lactococcus lactis strain comprising an inactivated phage infection protein obtained or obtainable by phage-hardening DSM 18874 against a bacteriophage which recognizes the phage infection protein (TC #3.A.1.155.1), such as the bacteriophage deposited as DSM 33304. The present invention provides a nisin-producing Lactococcus lactis strain comprising a phage infection protein with the polypeptide sequence as set forth in SEQ ID NO: 8, SEQ ID NO: 9, SEQ ID NO: 10 or SEQ ID NO: 11 or provides the Lactococcus lactis strain deposited as DSM 33302.

Given that Lactococcus lactis is a well-characterized, food-grade lactic acid bacterium (LAB) with generally recognized as safe (GRAS) status, the lactococcal strain provided herein may be advantageously used as starter culture in the food industry. The present invention provides a composition which comprises Lactococcus lactis strains disclosed herein, which can be used as starter culture. In the latter case, the composition may additionally comprise other starter bacteria for the fermentation of the food product. A skilled person in the art is able to select suitable starter bacteria based on the type of the food product. The present invention may be used in the preparation of food products including fermented food products, such as dairy products (including cheese), meat products or fermented plant products. Presence of nisin in combination with the sustained acidification can be used to reduce the risk of late-blowing in cheese and other fermented food products caused by spoilage by Gram-positive bacteria.

“Fermentation” in the methods of the present invention means the conversion of carbohydrates into alcohols or acids through the action of a microorganism. Preferably, fermentation in the methods of the invention comprises conversion of lactose to lactic acid.

An advantage of the present invention is the increased phage resistance, an important feature for dairy starter cultures.

In one embodiment, the composition may additionally comprise at least one strain of the genera selected from Lactobacillus, Streptococcus, Lactococcus and Leuconostoc, such as at least one strain of Lactobacillus bulgaricus and at least one strain of Streptococcus thermophilus or such as at least one strain of Lactococcus lactis, at least one strain of Leuconostoc mesenteroides subsp. cremoris.

The bacteria may be supplied to the industry either as frozen or freeze-dried cultures for bulk starter propagation or as so-called “Direct Vat Set” (DVS) cultures, intended for direct inoculation into a fermentation vessel or vat for the production of a fermented product, such as a fermented dairy like cheese. The starter culture composition is preferably in a frozen, dried or freeze-dried form, e.g. as a Direct Vat Set (DVS) culture. However, the composition may also be a liquid that is obtained after suspension of the frozen, dried or freeze-dried cell concentrates in a liquid medium such as water or PBS buffer. Where the composition of the invention is a suspension, the concentration of viable cells is in the range of 10⁴ to 10¹² cfu (colony forming units) per ml of the composition including at least 10⁴ cfu per ml of the composition, such as at least 10⁵ cfu/ml, e.g. at least 10⁶ cfu/ml, such as at least 10⁷ cfu/ml, e.g. at least 10⁸ cfu/ml, such as at least 10⁹ cfu/ml, e.g. at least 10¹⁰ cfu/ml, such as at least 10¹¹ cfu/ml.

The composition of the present invention may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectant are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

Provided herein is a DVS composition, preferably frozen or freeze-dried, comprising a nisin-producing Lactococcus lactis strain comprising an inactivated phage infection protein (TC #3.A.1.155.1), preferably Lactococcus lactis subsp. lactis or Lactococcus lactis subsp. cremoris or Lactococcus lactis subsp. lactis biovar diacetylactis.

Another aspect of the invention relates to a method of manufacturing a food or feed product comprising adding a starter culture composition as described herein to a food or feed product starting material and keeping the thus inoculated starting material under conditions where the lactic acid bacterium is metabolically active.

Useful food product starting materials include any material which is subjected to a lactic acid bacterial fermentation step such as milk, vegetable materials, meat products, fruit juices, must, doughs and batters. The fermented products, which are obtained by the method, include as typical examples dairy products such as cheese including fresh cheese products, and buttermilk.

Fermentation processes to be used in production of fermented milk products are well known and the person of skill in the art will know how to select suitable process conditions, such as temperature, oxygen, amount and characteristics of microorganism(s) and process time. Fermentation conditions are selected so as to carry out the present invention, i.e. to obtain a dairy product in solid or liquid form.

The present invention also includes a fermented dairy product which comprises a nisin-producing Lactococcus lactis strain comprising an inactivated phage infection protein (TC #3.A.1.155.1), preferably Lactococcus lactis subsp. lactis or Lactococcus lactis subsp. cremoris or Lactococcus lactis subsp. lactis biovar diacetylactis. The dairy product is for example cheese. The cheese may be a cheddar type cheese or a continental type cheese (e.g. Gouda, Danbo, Havarti, etc.). The term “cheese” refers to a product prepared by contacting milk, which may optionally be acidified, (e.g. by means of a lactic acid bacterial culture), with a coagulant, and draining the resultant curd. Cheeses and their preparation are described in “Cheese and Fermented Milk Foods”, by Frank V. Kosikowski. The term “cheese of the cheddar type” should be understood as cheeses of the types such as Cheddar, Territorials, American Cheddar, Monterey Jack and Colby, and/or cheeses made by a process which includes heating the curd to a temperature that does not exceed 45 degrees C. In the present context, cheese of the cheddar type is characterized by:
Fat in Dry matter: 10-60%
Humidity: 34-42%
Salt content: 1.5-2.5%
Cheddaring and subsequent Milling step
Salting after milling but before pressing
Pressing step

The term “cheese of the continental type” should be understood as cheeses of the types, such as Gouda, Danbo, Edam, St. Paulin, Raclette, Fontal etc. and/or cheeses made by a process which includes heating the curd to a temperature that does not exceed 45 degrees C. In the present context, cheese of the continental type is characterized by:
Fat in Dry matter: 10-60%
Water content: 35-57%
Water in Fat free cheese matter: 53-63%
Salt content: 1-3.5%
Pressing step during cheese manufacture process
Salting after pressing most often in a brine.

Included herein is a fermented dairy product, such as cheese, comprising a nisin-producing Lactococcus lactis strain DSM 33302 or a nisin-producing Lactococcus lactis strain comprising an phage infection protein with the polypeptide sequence as set forth in SEQ ID NO: 8, SEQ ID NO: 9, SEQ ID NO: 10 or SEQ ID NO: 11.

Furthermore, the present invention can also be useful in the manufacturing of nisin as food additive or as a pharmaceutical agent. Researches have verified its potential use for therapeutic purposes, for example in the treatment of atopic dermatitis, stomach ulcers and colon infections, respiratory tract infections caused by Staphylococcus aureus, and staphylococcal mastitis during lactation. (de Arauz et al., “Nisin biotechnological production and application: a review.” Trends in Food Science & Technology 20.3-4 (2009): 146-154).

Industrially, nisin can be manufactured via fermentation of fluid milk or whey by strains of the present invention Lactococcus lactis. The resulting fermentation broth is subsequently concentrated and separated, spray dried and milled to yield small particles. In view of the constant need to improve nisin yield and lower manufacturing costs, typically by optimize the media of cheaper cultivation substrates, the present invention can therefore be applied to further improve the economics of the production of nisin.

The present invention provides a method of obtaining nisin comprising culturing a nisin-producing Lactococcus lactis strain as disclosed in the present application under conditions which allow for nisin production. Such conditions can be readily determined by a skilled person in the art. More nisin can be isolated from the daughter strain compared to the mother strain due to the increase in nisin production. In one preferred embodiment, nisin production is evaluated according to the culturing conditions and methods as described in Example 2.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

Applicant deposited the Lactococcus lactis DSM 18874 on 2006-12-19 at Leibniz Institute DSMZ—Deutsche Sammlung von Mikroorganismen und Zellkulturen GmbH (DSMZ), Inhoffenstr. 7B, D-38124 Braunschweig and received the accession No.: DSM 18874.

Applicant deposited the Lactococcus lactis strain A on 2019-10-09 at Leibniz Institute DSMZ—Deutsche Sammlung von Mikroorganismen und Zellkulturen GmbH (DSMZ), Inhoffenstr. 7B, D-38124 Braunschweig and received the accession No.: DSM 33302.

Applicant deposited the c2-bacteriophage CHPC1242 on 2019-10-09 at Leibniz Institute DSMZ—Deutsche Sammlung von Mikroorganismen und Zellkulturen GmbH (DSMZ), Inhoffenstr. 7B, D-38124 Braunschweig and received the accession No.: DSM 33304.

### EXAMPLES

### Example 1.1 Obtaining Mutants from DSM 18874 as Mother Strain

A commercial product Lactococcus lactis DSM 18874 with good sensory and acidification profile in milk is provided as the mother strain. A genome analysis showed that it contains the phage infection protein with the polypeptide sequence of 901 aa as set forth in SEQ ID NO: 7.

To obtain mutants where pip is mutated, DSM 18874 was challenged with a bacteriophage belonging to the species of c2 type phages, deposited as DSM 33304 (CHPC1242).

The mutants were isolated on M17-1% lactose agar plates with 10 mM MgCl₂/CaCl₂ after plating 0.1 ml of an overnight culture of DSM 18874, grown in M17-1% lactose at 30° C., together with 0.1 ml of a lysate of phage DSM 33304 containing 1e09 phage particles per ml and 1 to 4 days of incubation at 30° C. until the appearance of colonies. For this, phages and host strain were mixed with 2.5 ml of a top agar solution (molten agar with half (0.75%) of the standard agar concentration kept at 46 to 50° C.), and poured on a bottom M17-1% lactose agar. Both bottom and top agar were containing 10 mM MgCl₂/CaCl₂. When the top agar was solidified incubation occurred with described conditions.

Among several mutants, four strains—A, B, C, and D—were three times colony-purified and retested in a standard plaque assay using phage DSM 33304 (CHPC1242). Phage resistance was confirmed, as no single plaques were observed whereas DSM 18874 showed plaques on M17-1% lactose/MgCl₂/CaCl₂ agar plates.

### Example 1.2 Pip Sequence in Mutant Strains

This example demonstrates that the mutants obtained by challenging the mother strain DSM 18874 with the c2 type phage contain mutations in pip.

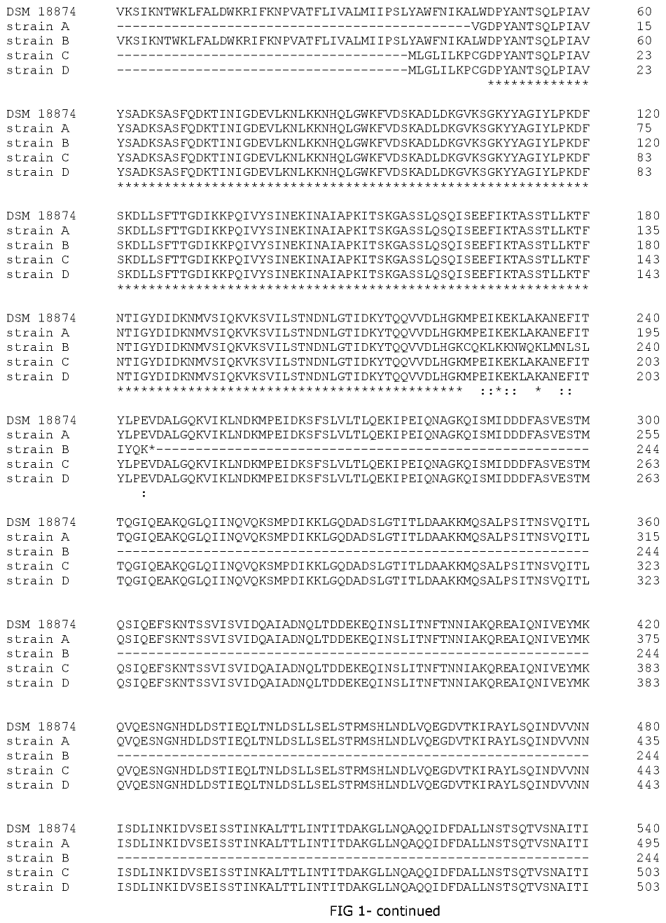

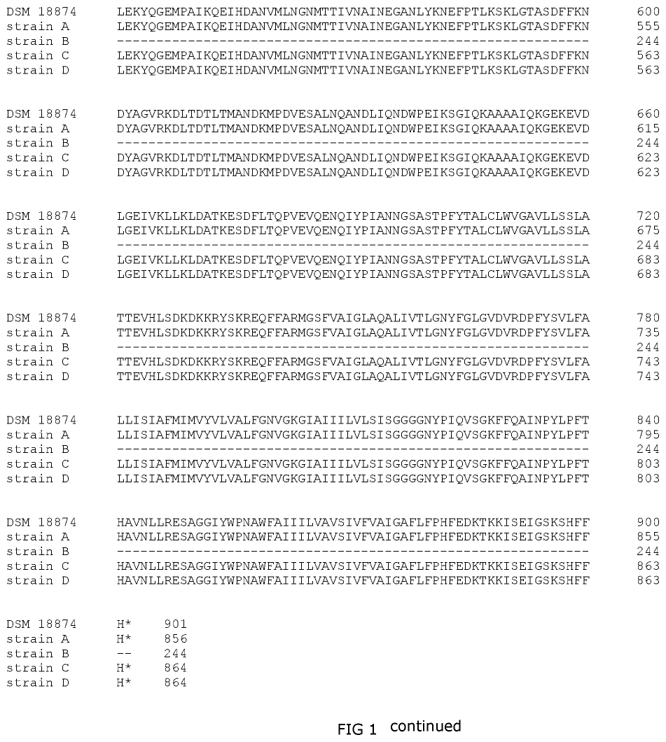

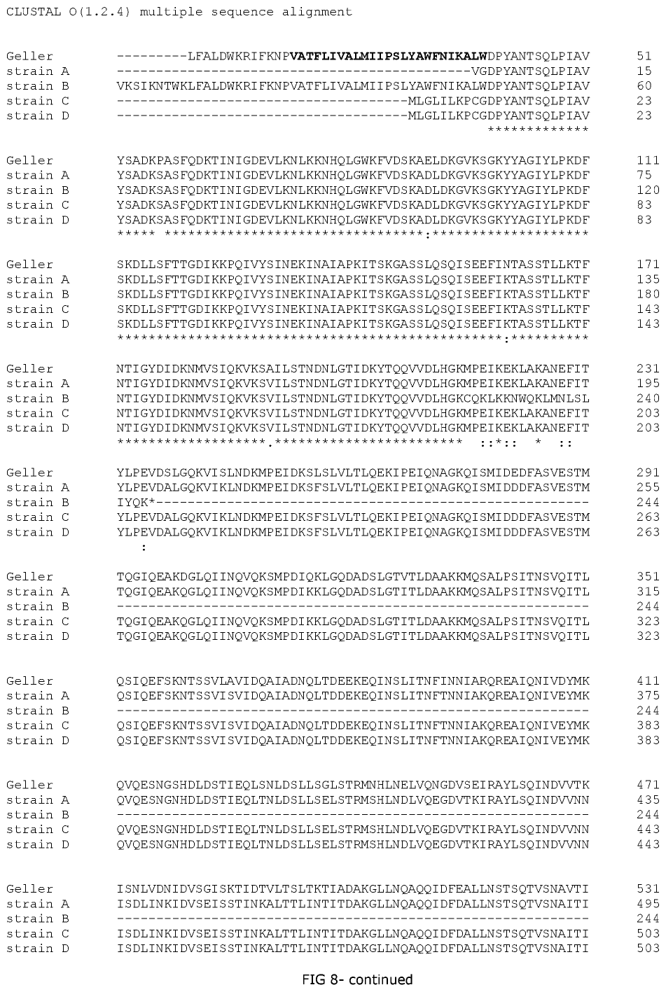

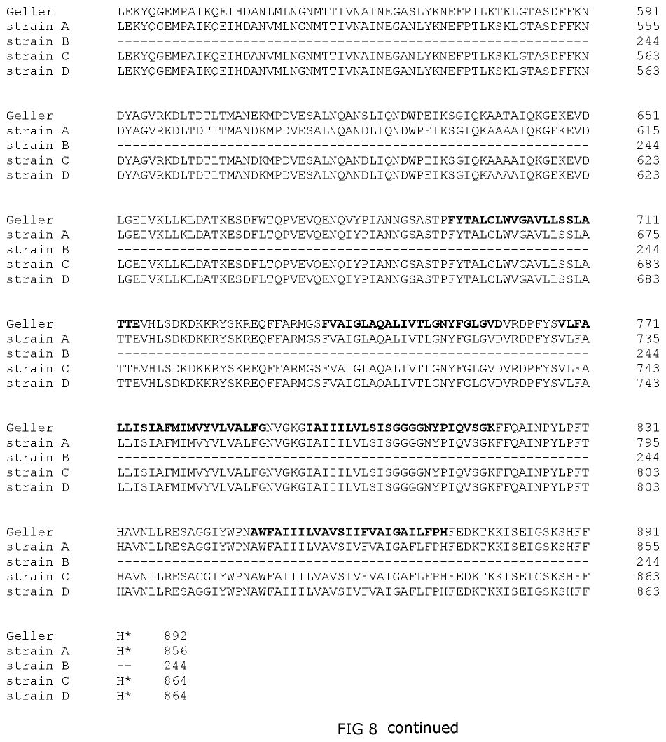

A genome analysis of mutant strains A, B, C, and D shows mutations within the pip gene. FIG. 1 shows the polypeptide sequences of Pip from the mutants compared to the mother strain DSM 18874. All four selected mutants A, B, C, and D show mutations within the phage infection protein. FIG. 8 depicts the alignment of four pip mutants A, B, C and D (SEQ ID NO: 8-11) with Pip polypeptide sequences reported by Geller (SEQ ID NO: 1). The predicted transmembrane domains at positions 14-38, 694-714, 738-760, 768-790, 796-819 or 849-873 are marked bold.

In strain A (DSM33302), the sequence shows an N-terminal truncation. The gene size of the pip gene of this strain is 2571 bps whereas the size of the wild type pip gene is 2706 bps. In strain C and strain D, an N-terminal truncation in pip is also present.

It is known that the phage infection protein is a membrane associated protein, acting as phage receptor for lactococcal phages of the c2 type. Within the first 60 residues of Pip a putative signal sequence and a hydrophobic, potential membrane-spanning region are located (Geller et al. 1993). A deletion of the first amino acids, as in mutants A, C, and D, would therefore most likely have a dramatic impact on functionality of the protein, explaining the phage resistance phenotype.

In strain B, the sequence shows a C-terminal truncation. The gene size of the pip gene of this strain is only 735 bps ending by a stop codon. Here, the largest part of Pip including all C-terminal membrane-spanning regions (Geller et al. 1993, J. Bac. Vol. 175, no. 17, p. 5510-5519) are missing, which has as well a huge impact on morphology and functionality of Pip.

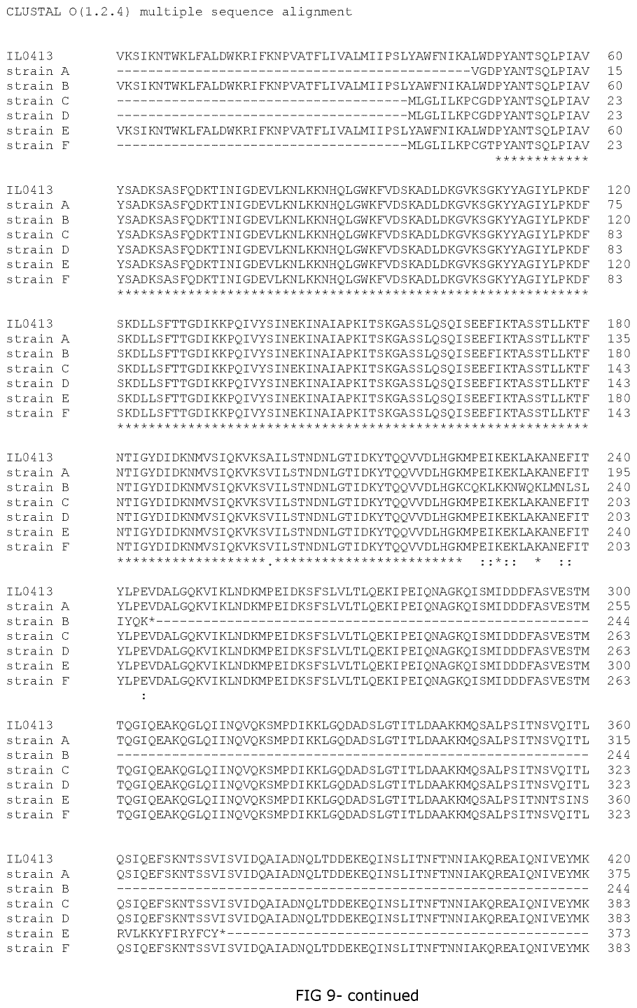

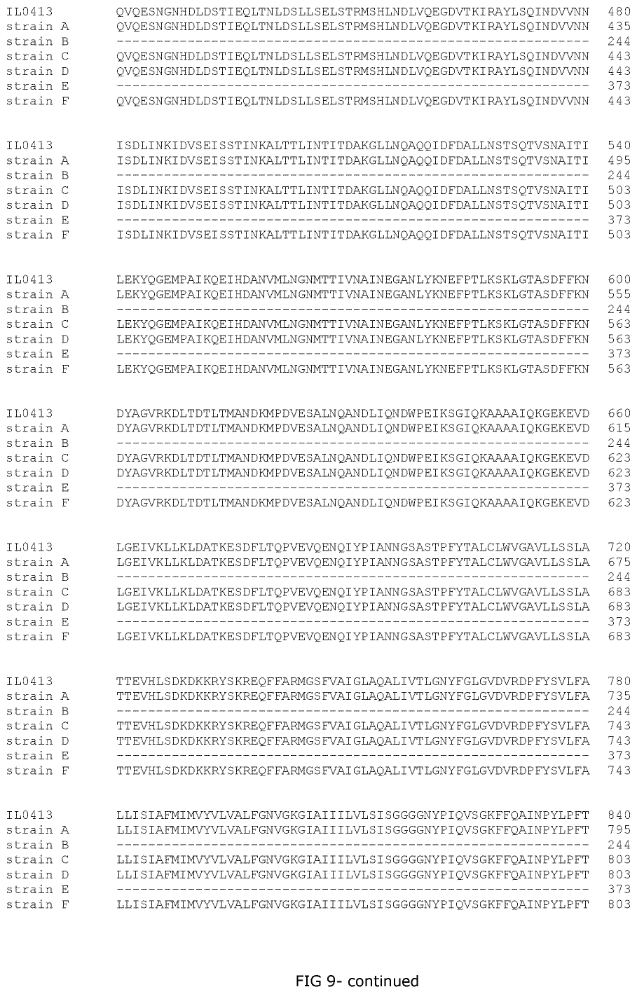

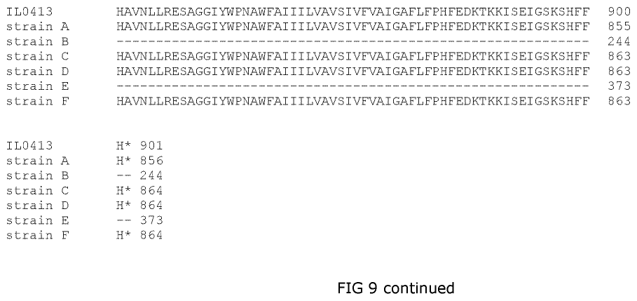

In a separate experiment, mutant strains E and F were obtained the same way as described in Example 1. The Pip sequences of stain E and F are shown in SEQ ID NO: 12 and 13, respectively. FIG. 9 shows a sequence alignment of strains A-F with the Pip polypeptide sequences of the IL1403 strain.

In strain E, the sequences shows a C-terminal truncation. The observed mutation was a deletion of 11 bps at position 1068 of the pip gene which leads to gene truncation. The remaining part of the pip gene has a size of 1122 nucleotides ending by a stop codon.

In strain F, the sequence shows an N-terminal truncation. The observed mutation was the introduction of an additional nucleotide C at position 147 of the pip gene leading to gene truncation at the beginning of the gene. The remaining part of the gene, downstream of the mutation, is an open reading frame of 2595 bps lacking the original promoter and ribosomal binding site of the pip gene.

### Example 2 Comparison of Nisin Production

This example demonstrates that the obtained pip mutants of nisin-producing strains exhibit increased nisin production.

Culturing Conditions

Lactococcus lactis strains DSM 18874, strains A, B, C, D (Example 1) and the nisin-sensitive indicator strain Wg2 (Lactococcus lactis subsp. cremoris; available as DSMZ 4367 from Leibniz-Institut DSMZ-Deutsche Sammlung von Mikroorganismen and Zellkulturen GmbH, Germany) were grown from a −80° C. stock in 180 μl M17+2% (w/v) glucose and 2% (w/v) lactose, incubated at 30° C. for a minimum of 18 hours. Of this outgrown culture, 20 μl was transferred to 1980 μl of M17 plus 2% (w/v) glucose and 2% (w/v) lactose and incubated for a minimum of 18 hours at 30° C. to ensure full outgrowth. Samples from strain A, B, C, D and DSM 18874 were centrifuged for 10 minutes at 4.000 rpm (Hettich Rotanta 46 RSC, Germany) and 300 μl of the supernatant was filtered through a 96 well 0.2 μm filter-plate from AcroPrep (Pall, USA). The pH of the filtered supernatant was measured, after which the pH was adjusted to pH 6.0 by the addition of 21-22 μl 0.25M NaOH.

Fractions of the supernatant were analyzed for nisin concentrations.

Nisin Measurement

A mixed standard containing 50 ng/ml in the injected solution of nisin A, Chrisin, Material #502095, Chr. Hansen A/S, was used for quantification of the nisin A amount in the samples.

The samples and standards were diluted hundred times using a BSA-buffer with internal standard (0.01 mg Bovine Serum Albumin, Sigma #A2153/0.5 ml formic acid, Thermo #28905/20 ml acetonitrile, Merck #83640.290/0.1 mg chloramphenicol, Sigma #C0378 dissolved in Milli-Q-Water to a total volume of 100 ml). The diluted samples and standards were placed in the autosampler at max. 8° C. and analyzed on a LC-MS/MS, Acquity I-class UPLC coupled to a Xevo TQ-XS Triple quadrupole mass spectrometer interfaced with a Z-spray ESI by injection of 2 ul (all from Waters, USA).

Gradient separation with time table: 0 min: 90% A/10% B; 0.20 min: 90% A/10% B; 3.50 min: 45% A/55% B; 3.70 min: 0% A/100% B; 5.45 min: 0% A/100% B; 5.55 min: 90% A/10% B; 6.35 min: 90% A/10% B, at 0.50 ml/min at 60° C. using a PLRP-S column, 300 Å, 2.1×150 mm, 3 μm, PL1912-3301 (Agilent Technologies, USA) using two mobile phases was employed. Mobile phase A was 0.05% formic acid in Milli-Q-Water and mobile phase B was 0.05% formic acid in acetonitrile.

Data collection on the mass spectrometer was done in positive electrospray ionization mode, Capillary 3 kV, Cone 40 V, Source Offset 30 V, Source Temperature 150° C., Desolvation Temperature 650° C., Cone Gas Flow 150 L/Hr, Desolvation Gas Flow 1100 L/h, Collision Gas Flow 0.25 mL/min, Nebulizer Gas Flow 6 Bar. Multiple Reaction Monitoring MRM used was (quant ion underlined): Nisin A: 2.00-2.70 min: 671.7>790.0; 671.7>806.8; 671.7>811.2, all @ 16 eV, cone 45 V; Nisin Z: 2.75-3.30 min: 667.2>739.1; 667.2>805.4; 667.2>739.1; all @ 16 eV, cone 45 V; Chloramphenicol: 2.75-3.30 min: 321.3>152.0; 321.3>257.2; all @ 15 eV, cone 25 V.

The peak height of Nisin A and Nisin Z respectively, was used to calculate the concentration in the supernatant (ng/ml).

Table 1 shows the pH of the supernatant of the tested strains and nisin A measurements. Nisin Z was not produced by the strains.

TABLE 1

| | pH supernatant after | nisin A | nisin A |
|---|---|---|---|
| Strain | filtering | (peak height) | ng/ml |
| DSM 18874 | 4.3 | 896176 | 35.2 |
| A | 4.3 | 2344701 | 89.9 |
| B | 4.3 | 1834089 | 69.5 |
| C | 4.3 | 1616436 | 61.6 |
| D | 4.4 | 1741151 | 67.8 |

As shown in Table 1, the mutant strains A, B, C, D all produced higher nisin level than the mother strain.

Nisin production of strains E and F were also compared with the morther strain DSM 18874. Strain E and F produce about 48.5% and 50.6% more nisin that the mother strain, respectively (data not shown).

### Example 3 Comparison of Nisin Production in Fermented Milk Product

Nisin-producing cultures may be added to a starter culture for fermented food products such as fermented dairy products. Strain A, the strain with the highest nisin production from Example 1 was chosen to demonstrate this.

In detail, nisin-producing strain A and DSM 18874 were tested in two different setups: (1) as single strains without any starter, and (2) together with the starter culture F-DVS C501 (available from Chr. Hansen, Denmark). C501 contains the species Lactococcus lactis subsp. cremoris, Lactococcus lactis subsp. lactis, Lactococcus lactis subsp. lactis biovar. diacetylactis, and Leuconostoc, a product normally used for continental cheese production.

Fermentation was carried out at 30° C. in commercial semi-skimmed milk containing 1.5% fat, 3.5% protein (ARLA 24 Frisktappet letmælk, Arla Foods, Denmark). 0.2 g/v % yeast extract was added in bottles containing only the nisin producing strain (setup (1)). The nisin producing strains A and DSM 18874 were inoculated from Direct Vat Set (DVS) material, and standardized according to the CFU at 5.5E6 CFU/ml. F-DVS C501 was inoculated as DVS material at the recommended dose at 9.6 g/100 L. After 18 hours of fermentation, samples were frozen down at −20° C. until measured by HPLC-MS/MS. All samples were run in at least duplicates.

Nisin Measurement

A mixed standard containing 50 ng/ml in the injected solution of each nisin A (Chrisin, Material #502095, Chr. Hansen A/S) and nisin Z (Chrisin C, Material #671535, Chr. Hansen A/S).

Samples were extracted and diluted 1 to 29 (30×) with BSA Extraction buffer and kept overnight at 5′C. Extraction buffer: 0.01 mg BSA dissolved in 100 ml 20% acetonitrile in MQW also containing 0.50% formic acid, and then 1.5 ml nisin Q stock solution per 100 ml BSAeExtraction buffer.

The diluted samples were placed in the autosampler at max. 8° C. and analyzed on a LC-MS/MS, Acquity I-class UPLC coupled to a Xevo TQ-XS Triple quadropole mass spectrometer interfaced with a Z-spray ESI, all from Waters, by injection of 2 ul.

Gradient separation with time table: 0 min: 90% A/10% B; 0.20 min: 90% A/10% B; 3.50 min: 45% A/55% B; 3.70 min: 0% A/100% B; 5.45 min: 0% A/100% B; 5.55 min: 90% A/10% B; 6.35 min: 90% A/10% B, at 0.50 ml/min at 60° C. using a PLRP-S column, 300 Å, 2.1×150 mm, 3 μm, PL1912-3301, Agilent Technologies, using two mobile phases was employed. Mobile phase A was 0.05% Formic acid, Thermo #28905, in Milli-Q-Water and mobile phase B was 0.05% Formic acid, Thermo #28905, in Acetonitrile, Merck #83640.290.

Data collection on the mass spectrometer was done in positive electrospray ionization mode, Capillary 3 kV, Cone 40 V, Source Offset 30 V, Source Temperature 150° C., Desolvation Temperature 650° C., Cone Gas Flow 150 L/Hr, Desolvation Gas Flow 1100 L/h, Collision Gas Flow 0.25 mL/min, Nebuliser Gas Flow 6 Bar. MRM used was (quant ion underlined): Nisin A: 2.00-2.70 min: 671.7>790.0; 671.7>806.8; 671.7>811.2, all @ 16 eV, cone 45 V; Nisin Z: 2.75-3.30 min: 667.2>739.1; 667.2>805.4; 667.2>739.1; all @ 16 eV, cone 45 V.

The peak area of Nisin A and Nisin Z respectively, was used to calculate the concentration.

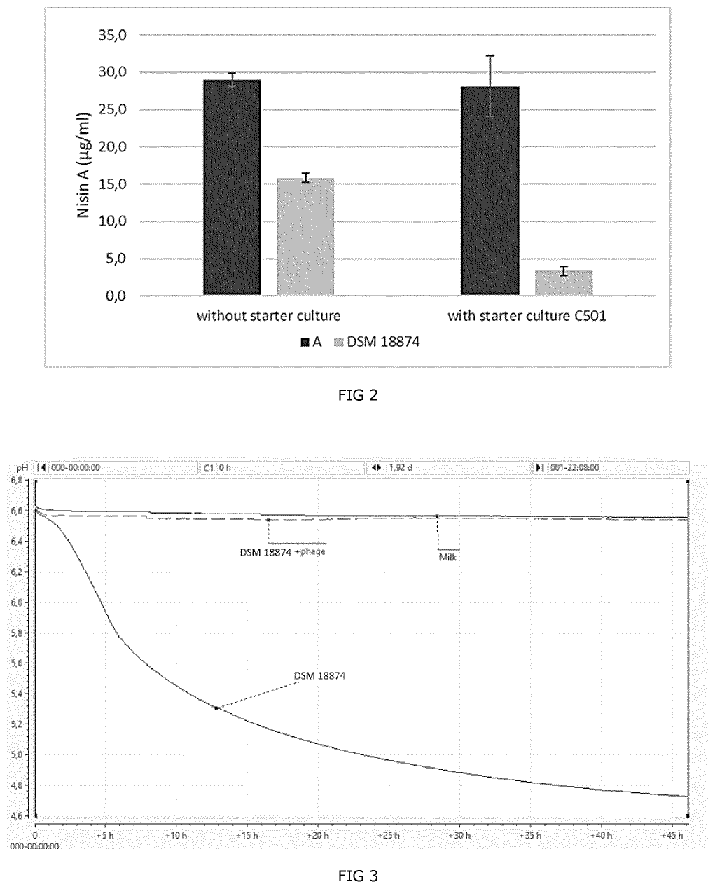

FIG. 2 demonstrates that strain A produces significantly more nisin A than the mother strain. This is the case both when the nisin producing starter culture is fermenting alone, as well as together with the starter culture C501. Furthermore, it was surprisingly observed that the nisin level produced by the mutant in the presence of the starter culture does not differ much from when it is used alone; this is not the case for the mother strain DSM 18874.

This example shows the nisin production level of strain A is higher in fermented milk, with and without starter culture, compared to the mother strain.

### Example 4 Acidification Profile

DSM 18874 and strains A, B, C and D were evaluated for phage resistance and acidification activity in milk, made by reconstituting low fat skim milk powder at a level of dry matter of 9.5% in distilled water and pasteurizing at 99° C. for 30 min, with and without the presence of c2 type phage DSM 33304 (CHCC1242).

The strains were inoculated in the milk 1% from overnight cultures (overnight cultures in M17 with 1% lactose) and incubated for 46 hours at 30° C.

The pH was monitored continuously during the fermentation using a PC logger (AAC-2 PC logger; Intab Interface Teknik AB, Stenkullen, Sweden). PH curves were visualized with the EasyView software (Intab Interface Teknik AB, Stenkullen, Sweden).

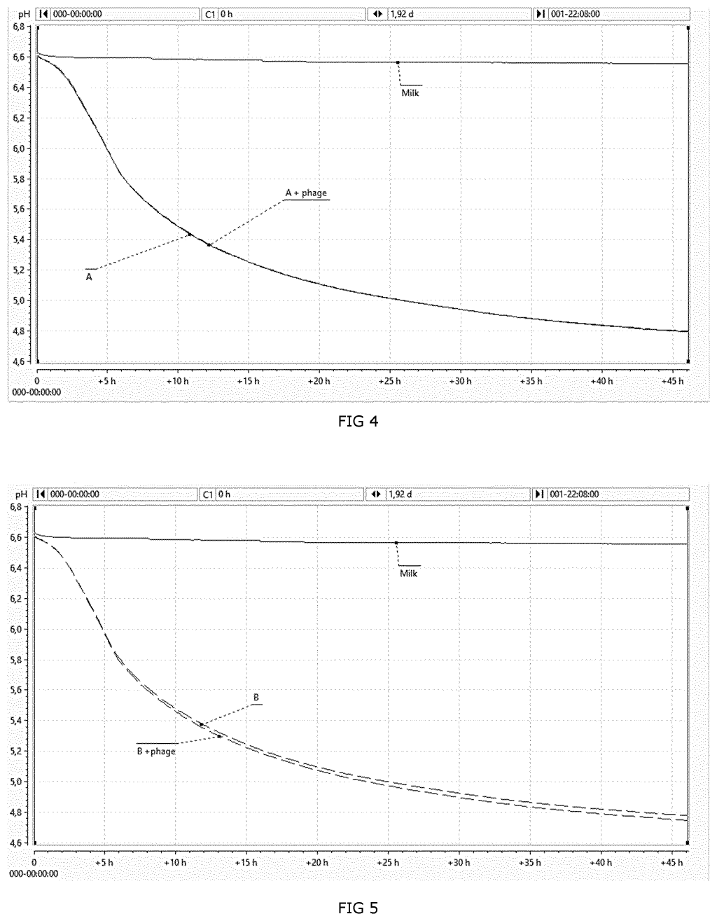

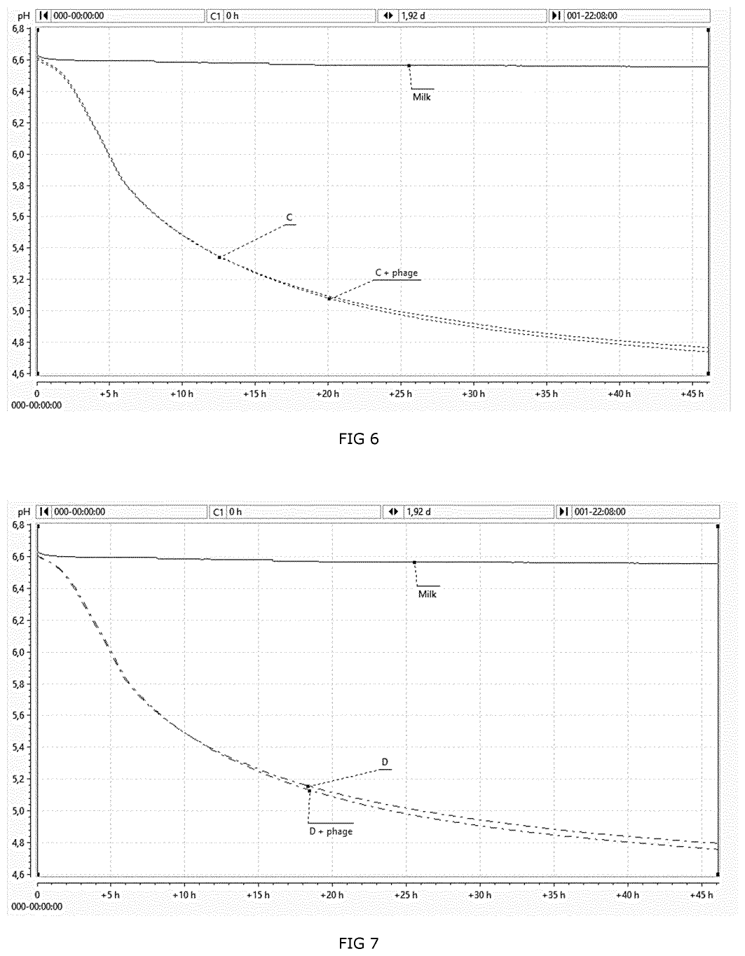

Acidification profiles for the strains are shown in FIGS. 3-7. As shown, whereas the acidification of mutants A, B, C and D was not affected by the presence of phage, thus confirming the phage resistance phenotype, acidification of the mother strain DSM 18874 was completely inactivated by phage DSM 33304.

Advantageously, the figures show that the acidification activity of the four mutants is very similar to the activity of mother strain, indicating that the introduction of the mutation within the pip gene did not show undesirable adverse effects such as reduced acidification activity. Given acidification activity in milk is one of the most relevant parameters for dairy starter cultures, the mutant strains are also applicable in the industry as the mother strain.

1. A method for increasing nisin production in nisin-producing Lactococcus lactis, comprising:
providing one or more nisin-producing Lactococcus lactis strains which express the phage infection protein (TC #3.A.1.155.1) as mother strain,
mutating of the mother strain gene(s) which encode the phage infection protein or regulate the expression of gene(s) which encode the phage infection protein,
obtaining one or more Lactococcus lactis mutants in which the phage infection protein is inactivated, and
selecting from the obtained mutants one or more Lactococcus lactis daughter strains whose nisin production is increased compared to the mother strain.

2. The method according to claim 1, wherein the mutating step is carried out by deleting fully or partially the gene(s) which encodes the phage infection protein or regulate the expression of gene(s) which encode the phage infection protein.

3. The method according to claim 1, wherein the mutating step is carried out by phage hardening against a bacteriophage which recognizes the phage infection protein, for example a c2 type bacteriophage.

4. The method according to any one of the previous claims, wherein the nisin-producing Lactococcus lactis produces nisin A or nisin Z.

5. The method according to any one of the previous claims, wherein the nisin-producing Lactococcus lactis is Lactococcus lactis subsp. lactis or Lactococcus lactis subsp. cremoris or Lactococcus lactis subsp. lactis biovar diacetylactis.

6. The method according to any one of the previous claims, wherein the nisin production is measured by growing the strain in a condition which allows for nisin production.

7. The method according to any one of the preceding claims, wherein the mother strain is the Lactococcus lactis deposited as DSM 18874.

8. The method according to claim 3, wherein the bacteriophage is the c2 type phage CHPC1242 deposited as DSM 33304.

9. A nisin-producing Lactococcus lactis strain obtained or obtainable by any one of the preceding claims.

10. A nisin-producing Lactococcus lactis strain comprising an inactivated phage infection protein (TC #3.A.1.155.1), preferably Lactococcus lactis subsp. lactis or Lactococcus lactis subsp. cremoris or Lactococcus lactis subsp. lactis biovar diacetylactis.

11. The nisin-producing L. lactis strain according to claim 10, preferably Lactococcus lactis subsp. lactis or Lactococcus lactis subsp. cremoris or Lactococcus lactis subsp. lactis biovar diacetylactis, comprising a phage infection protein, wherein the protein lacks fully or substantially one, two, three, four, five or six of the predicted transmembrane domains corresponding to amino acids 14-38, 694-714, 738-760, 768-790, 796-819 or 849-873 of Pip as set forth in SEQ ID NO: 1, or comprising a phage infection protein, wherein the protein lacks fully or substantially one, two, three, four or five of the predicted transmembrane domains corresponding to amino acids 694-714, 738-760, 768-790, 796-819 or 849-873 of Pip as set forth in SEQ ID NO: 1.

12. The nisin-producing Lactococcus lactis strain according to claim 10, comprising an inactivated phage infection protein obtained or obtainable by phage-hardening against a bacteriophage which recognizes the phage infection protein (TC #3.A.1.155.1).

13. The nisin-producing Lactococcus lactis strain according to claim 10, which is deposited as DSM 33302 or which is a nisin-producing Lactococcus lactis strain comprising an phage infection protein with the polypeptide sequence with at least 90% homology of the sequences as as set forth in SEQ ID NO: 8, SEQ ID NO: 9, SEQ ID NO: 10 or SEQ ID NO: 11.

14. A food, preferably fermented dairy products, or a composition comprising a nisin-producing Lactococcus lactis strain according to any one of claims 9-13.

15. A method to obtain nisin comprising culturing the nisin-producing Lactococcus lactis strain according to any one of claims 9-13 under a condition which allows for nisin production, and optionally isolating therefrom nisin.
